(19) United States
(12) Patent Application Publication
(10) Pub. No.: US 2022/0102012 A1
(43) Pub. Date: Mar. 31, 2022

(54) SYSTEMS AND METHODS FOR PREDICTIVE MODELING OF PEOPLE MOVEMENT AND DISEASE SPREAD UNDER COVID AND PANDEMIC SITUATIONS

(71) Applicant: Arizona Board of Regents on Behalf of the University of Arizona, Tucson, AZ (US)

(72) Inventors: Young-Jun Son, Tucson, AZ (US); Saurabh Jain, Tucson, AZ (US); Bijoy Dripta Barua Chowdhury, Tucson, AZ (US); Md Tariqul Islam, Tucson, AZ (US); Yijie Chen, Tucson, AZ (US)

(21) Appl. No.: 17/491,215

(22) Filed: Sep. 30, 2021

Related U.S. Application Data
(60) Provisional application No. 63/085,933, filed on Sep. 30, 2020.

(51) Int. Cl. G16H 50/80; G16H 50/50; G16H 50/30; G06F 16/29

(52) U.S. Cl. CPC G16H 50/80; G06F 16/29; G16H 50/30; G16H 50/50

(57) ABSTRACT

Systems and methods are described for agent-based simulation of each individual's movements in order to monitor the propagation of a disease. An agent-based simulation model has been exemplarily constructed, which is mainly comprised of two parts: student mobility model and disease propagation model. In the student mobility model, movements of students are modeled based on the GIS map (viz. routes, distances) and their daily schedules (e.g. dorms and classrooms/buildings). The disease propagation model represents students' health status (viz. susceptible, pre-symptomatic, asymptomatic, quarantine, isolation, and recovered) based on different factors such as the number of infected students attending the class or living in a dorm, classroom/dorm features (e.g. size, humidity, ventilation), probabilities of disease transmissions (e.g. droplet, airborne) in classrooms based on a dose-response model, probabilities of disease transmissions in dorms based on cohort studies, and mask wearing condition and effectiveness.

### REFERENCE TO RELATED APPLICATIONS

This application claims the benefit of U.S. Provisional Application No. 63/085,933, filed on Sep. 30, 2020, the entire contents of which are incorporated herein by reference.

### FIELD OF THE INVENTION

The present invention relates to computer-implemented systems and methods for real-time surveillance, analysis, and mapping of populations at risk of diseases such as COVID-19 using a consolidated technological platform.

### BACKGROUND OF THE INVENTION

Diseases like COVID-19 have created significant viral spread and stress among clustered populations that are required to interact in physical locations, like university campuses or similar campus-like environments, e.g. senior living systems, jails, prisons, residential treatment facilities etc. Disease transmission, contact tracing, and mitigation of infection spread are difficult to manage when it is difficult to track population movement and interaction. Moreover, it is difficult to determine, in real-time, events that increase the risk of disease transmission, such as lack of masks, pinch-points, and crowding, inadequate building design and facilities operations, such as toilet plumes, inadequate ventilation, lack of operable windows.

Since January 2020, the severe acute respiratory syndrome COVID-19 disease has spread rapidly and become a worldwide pandemic which forced people to stay at home and self-quarantine to avoid close contacts in order to stop disease transmission. Therefore, many researchers have become concerned about re-opening of high schools and universities which may cause a second wave of COVID-19 pandemic. An agent-based model is developed in this study to evaluate contacts, layout, entrance/exit rules for indoor movements, and campus-wide mobility, disease propagation, and testing policy under a variety of scenarios, including different disease transmission modes, percentage of mask-wearing, percentage of in-person on-campus classes, and percentage of dorm room sharing.

As of Aug. 3, 2020, more than 17.5 million cases of coronavirus disease 2019 (COVID-19) and 680,000 deaths had been reported worldwide. Many universities in the US are planning to reopen campuses with in-person or hybrid classes during the academic year 2020-2021. To evaluate and establish effective measures, many universities have formed campus re-entry task forces comprised of people from public health, engineering, data analytics. This analysis focuses on the evaluation of decisions pertaining to the classroom policies (e.g. entry/exit policies, seating arrangement, capacity assignment, and class schedules), considering students contacts and physical distancing in the classrooms. This analysis would help the university stakeholders to take safe and informed decisions for conducting in-person classes and other university activities.

As such, there is a need in the art for a system that performs evaluation of decisions pertaining to the classroom policies (e.g. entry/exit policies, seating arrangement, capacity assignment, and class schedules), considering students contacts and physical distancing in the classrooms. This analysis would help the university stakeholders to take safe and informed decisions for conducting in-person classes and other university activities.

### SUMMARY OF THE INVENTION

Modeling and simulation of the classroom requires incorporation of realistic movements of the students into the classroom, while also maintaining the physical distancing under the pandemic situations. In this analysis, agent-based simulation has been utilized for simulation of each individual's movements in Anylogic 8.5. Furthermore, for students to maintain physical distancing policy, a pedestrian library with an embedded social force model has been used (FIG. 1). Different scenarios for entry-exit policies have been considered under the utilization of route choice models (e.g. Cross-Nested Logit, Probit and Logit Kernel model), whereas resource selection models have been utilized for the seat selection process by the students.

In one embodiment, an agent-based simulation model has been exemplarily constructed, which is mainly comprised of two parts: student mobility model and disease propagation model. In the student mobility model, movements of students are modeled based on the GIS map (viz. routes, distances) and their daily schedules (e.g. dorms and classrooms/buildings). The disease propagation model represents students' health status (viz. susceptible, pre-symptomatic, asymptomatic, quarantine, isolation, and recovered) based on different factors such as the number of infected students attending the class or living in a dorm, classroom/dorm features (e.g. size, humidity, ventilation), probabilities of disease transmissions (e.g. droplet, airborne) in classrooms based on a dose-response model, probabilities of disease transmissions in dorms based on cohort studies, and mask wearing condition and effectiveness.

In certain embodiments, the agent-based simulation model has been exemplarily constructed using Anylogic 8.5, available from the AnyLogic Company at https://www.anylogic.com/blog/anylogic-8-5-2/, which is mainly comprised of two parts: student mobility model and disease propagation model. In the student mobility model, movements of students are modeled based on the GIS map (viz. routes, distances) and their daily schedules (e.g. dorms and classrooms/buildings). The disease propagation model represents students' health status (viz. susceptible, pre-symptomatic, asymptomatic, quarantine, isolation, and recovered) based on different factors such as the number of infected students attending the class or living in a dorm, classroom/dorm features (e.g. size, humidity, ventilation), probabilities of disease transmissions (e.g. droplet, airborne) in classrooms based on a dose-response model, probabilities of disease transmissions in dorms based on cohort studies, and mask wearing condition and effectiveness. The airborne transmission model employed in the analysis is based on models that consider classroom volume, mask effectiveness, and ventilation condition as variables. The droplet transmission model employed in the analysis considers the contact times and frequencies in 0-3 feet and 3-6 feet. In the analysis, the contact times and frequencies are estimated based on the classroom size and occupancy level. The dose-response model is used to calculate the infectious risk based on the virus amount inhaled by every susceptible student. The disease propagation model also considers the probability of students becoming symptomatic or asymptomatic after getting infected along with the probabilistic pre-symptomatic period (incubation period) and the virus shedding rate.

In other embodiments, the present invention comprises systems and methods for predictive modeling, where a computer receives facility parameters, agent parameter settings, and agent generation data for a plurality of agents. The computer then calculates routing and seating policies for the plurality of agents and determines movement based on the self-consciousness of the agents, the force of other agents, and the force from the environment on the plurality of agents. The computer then determines an exit path restriction policy or a zonal policy for an enclosed area that minimizes the risk of disease propagation for the plurality of agents

In those embodiments, execution of the constructed agent-based simulation provides realistic animation of the movement of students as well as statistics for student's interactions. Statistics from two perspectives namely, risk and logistics were reported from the simulation, which would facilitate informed decision making. Risk was evaluated in the terms of average contact numbers as well as average contact time within two distance ranges (viz. 0-3 feet, and 3-6 feet). Moreover, the logistics for safe operations of in-person class were reported based on the exit times for all students to exit the class. FIG. 2 provides the results for reduction in average contact numbers and average contact duration when the zone-based exit policy is implemented. FIG. 3 shows a significant reduction in the risk metrics for different levels of occupancies of the classroom.

FIGS. 4A through 4C show the simulation of disease transmission in a two-week period. Exemplarily, the model is focused on the mask-wearing percentage and how will it reduce the disease spread. The considered conditions include 1) 5 classes per day on average taken by students, 2) a maximum classroom occupancy of 50% capacity, 3) non-sharing of dorm rooms with others, and 4) varying pandemic conditions on the date of campus re-opening (e.g. percentages of pre-symptomatic, asymptomatic, and recover). According to the simulation, if 100% of students are wearing a mask, it can reduce 90% of newly infected cases compared with 0% of students wearing a mask.

The simulation model will allow public health personnel and decision makers to evaluate different policies (e.g. reduction of class-size, shutdown of some buildings, and durations of quarantine) in terms of disease spreads (e.g. new infected cases) based on dynamically updated situations after campus re-opening.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a graphical depiction of the results of the predictive modeling of the present invention, showing the agent-based simulation model of the classroom;

FIG. 2 is a chart showing the results for zone-based vs. non-zone-based exit policy, in accordance with an embodiment of the present invention;

FIG. 3 is a chart showing the results of a reduction in risk metrics for different occupancy levels, in accordance with an embodiment of the present invention;

FIG. 4A is an exemplary graphical representation of the simulation parameters for the predictive model;

FIG. 4B is an exemplary graphical representation of a GIS map and students' movements for the predictive model;

FIG. 4C is an exemplary graphical representation the disease propagation statistics for two weeks and results for the predictive model;

FIG. 5 is an exemplary embodiment of the hardware of the predictive modeling system;

FIG. 6 shows a flowchart of the high-level predictive modeling performed by an exemplary embodiment of the invention;

FIG. 6 shows a diagram of the disease propagation states that are assigned to agents in the predictive modeling performed by an exemplary embodiment of the invention;

FIG. 7 is a flowchart outlining an exemplary algorithm of the predictive modeling performed by the present invention;

FIG. 8A is a graph showing new infected case reduced percentage (comparing with 0% mask condition) under different mask wearing percentage campus-wide;

FIG. 8B is a graph showing new infected case reduced percentage (comparing with 0% mask condition) under different mask wearing percentage in a classroom;

FIG. 9A is a graph showing the percentage of test positive cases among infected agents under different test policies where the initial infection rate is 0.5%;

FIG. 9B is a graph showing the percentage of test positive cases among infected agents under different test policies where the initial infection rate is 5%;

FIG. 10A is a graph of the estimation of the R₀ value of different vaccination rates under a Stage 1 reopening;

FIG. 10B is a graph of the estimation of the R₀ value of different vaccination rates under a Stage 2 reopening;

FIG. 11A is a graph comparing an exemplary simulation prediction and University of Arizona's main campus actual test results from Sep. 14, 2020 to Oct. 30, 2020.

FIG. 11B is a graph comparing an exemplary simulation prediction to University of Arizona's main campus actual test results from Jan. 11, 2021 to Feb. 26, 2021.

FIG. 12 shows a diagram of the mobility states that are assigned to agents in the predictive modeling performed by an exemplary embodiment of the invention;

FIG. 13 is a graph of exemplary campus COVID-19 transmission predictions for a given week based on the predictive modeling performed by an exemplary embodiment of the invention;

FIG. 14 is a graph estimating the Positive state for a given week based on the predictive modeling performed by an exemplary embodiment of the invention;

FIG. 15 is pedestrian flowchart, where agent movement logic is presented with the help of different pedestrian library blocks;

FIG. 16 is a simulation based on predictive modeling of different policy implementations that were tested in different classroom settings;

FIG. 17 is a flowchart of the input, methods, and output of the predictive modeling software in accordance with an embodiment of the present invention;

FIG. 18 is a diagram of the physical distancing states that are assigned to agents in the predictive modeling performed by an exemplary embodiment of the invention;

FIG. 19 is a simulation using the predictive modeling of the present invention that shows an exit time and risk parameters dashboard for a collaborative classroom setting;

FIG. 20 is a dashboard view of the average contact time & average contact number for an individual agent in accordance with an embodiment of the present invention;

FIG. 21A shows graphical representation of the different force components of the social force model;

FIG. 21B shows graphical representation of the different force components of the social force model;

FIG. 22A is a graphical representation of physical distancing, as it is analyzed by an exemplary embodiment of the predictive model of the present invention;

FIG. 22B is a graphical representation of physical distancing, as it is analyzed by an exemplary embodiment of the predictive model of the present invention;

FIG. 22C is a graphical representation of physical distancing, as it is analyzed by an exemplary embodiment of the predictive model of the present invention;

FIG. 23 is a flowchart depicting physical distancing and deadlock resolution (human intervention);

FIG. 24A is a graphical representation of no deadlock as it is analyzed by an exemplary embodiment of the predictive model of the present invention;

FIG. 24B a graphical representation of deadlock without violation of physical distancing as it is analyzed by an exemplary embodiment of the predictive model of the present invention;

FIG. 25 is a graphical representation of the seat labeling procedure used by an exemplary embodiment of the present invention;

FIG. 26A is a flowchart showing the seat sorting component of the seating policy of an exemplary embodiment of the present invention;

FIG. 26B is a flowchart showing the seat selection component of the seating policy of an exemplary embodiment of the present invention;

FIG. 27A is a graphical representation of SD seat penalization and seat selection for different door settings;

FIG. 27B a graphical representation of SD seat penalization and seat selection for different door settings;

FIG. 28 is a boxplot showing the average exit time for different simulation configurations;

FIG. 29A is a boxplot showing the average exposure duration for different simulation configurations;

FIG. 29B is a boxplot showing the average exposure duration for different simulation configurations;

FIG. 29C is a boxplot showing the average exposure duration for different simulation configurations;

FIG. 29D is a boxplot showing the average exposure duration for different simulation configurations;

FIG. 30A is a boxplot showing the average contact number for different simulation configurations;

FIG. 30B is a boxplot showing the average contact number for different simulation configurations;

FIG. 31A is a boxplot showing the average contact number and exposure duration by varying physical distancing rule follower percentage;

FIG. 31B is a boxplot showing the average contact number and exposure duration by varying physical distancing rule follower percentage;

FIG. 32A is a graph showing the average exposure duration for a traditional classroom layout for two distance ranges under different occupancy levels;

FIG. 32B is a graph showing the average exposure duration for a collaborative classroom layout for 0-3 feet under different occupancy levels;

FIG. 32C is a graph showing the average exposure duration for a traditional classroom layout for 3-6 feet under different occupancy levels;

FIG. 32D is a graph showing the average exposure duration for a traditional classroom layout for 3-6 feet under different occupancy levels; and

FIG. 32E is a graph showing the average exposure duration for a traditional classroom layout for two distance ranges under different occupancy levels.

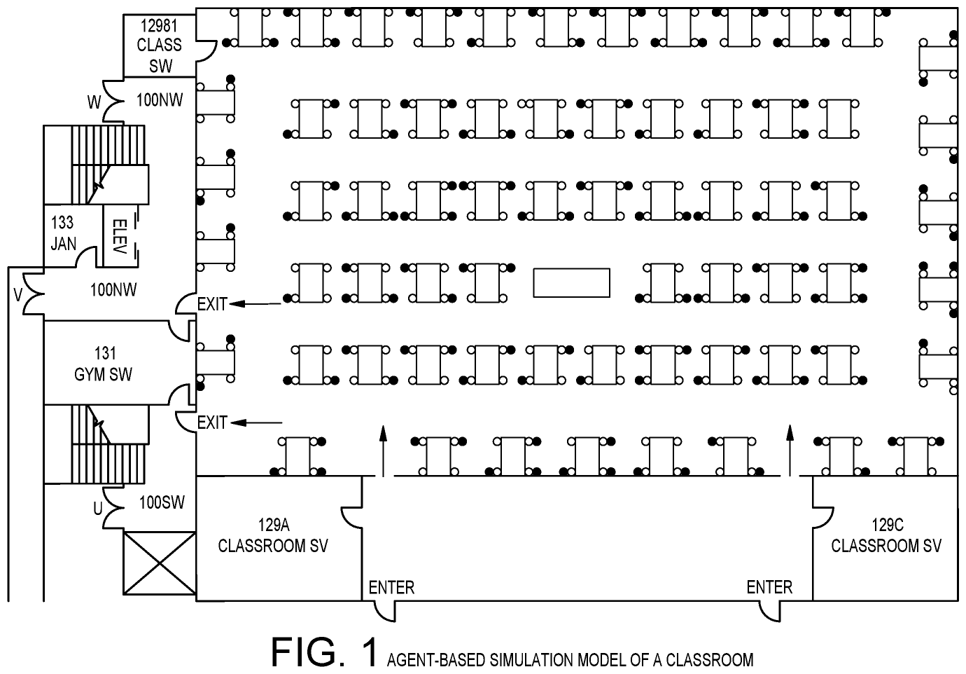

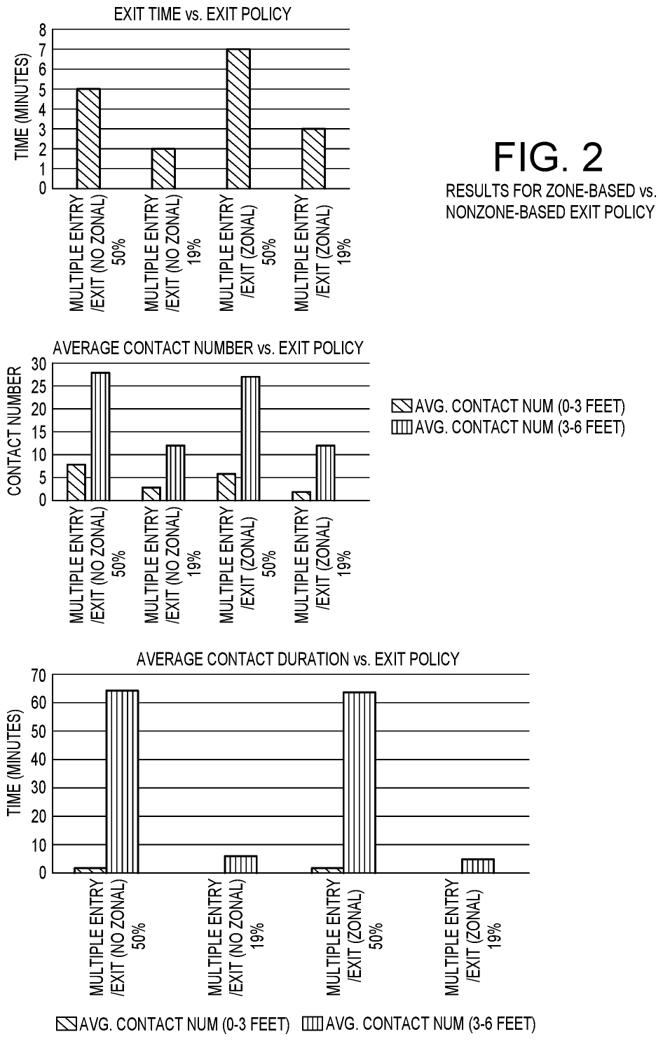

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

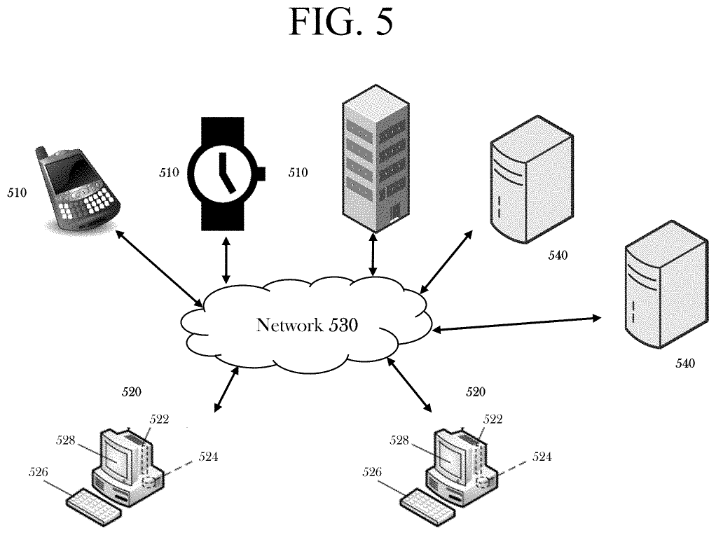

FIG. 5 is an exemplary embodiment of the predictive modeling system. In the exemplary system 500, one or more peripheral devices/locations 510 are connected to one or more computers 520 through a network 530. Examples of peripheral devices/locations 510 include smartphones, networked buildings, wearables devices, GPS devices, infrared sensors, servers with databases that contain a user's personal data, and any other devices that collect data that can be used to collect location and health data that are known in the art. The network 530 may be a wide-area network, like the Internet, or a local area network, like an intranet. Because of the network 530, the physical location of the peripheral devices/locations 510 and the computers 520 has no effect on the functionality of the hardware and software of the invention. Both implementations are described herein, and unless specified, it is contemplated that the peripheral devices/locations 510 and the computers 520 may be in the same or in different physical locations. Communication between the hardware of the system may be accomplished in numerous known ways, for example using network connectivity components such as a modem or Ethernet adapter. The peripheral devices/locations 510 and the computers 520 will both include or be attached to communication equipment. Communications are contemplated as occurring through industry-standard protocols such as HTTP or HTTPS.

Each computer 520 is comprised of a central processing unit 522, a storage medium 524, a user-input device 526, and a display 528. Examples of computers that may be used are: commercially available personal computers, open source computing devices (e.g. Raspberry Pi), commercially available servers, and commercially available portable device (e.g. smartphones, smartwatches, tablets). In one embodiment, each of the peripheral devices/locations 510 and each of the computers 520 of the system may have software related to the system installed on it. In such an embodiment, system data may be stored locally on the networked computers 520 or alternately, on one or more remote servers 540 that are accessible to any of the peripheral devices/locations 510 or the networked computers 520 through a network 530. In alternate embodiments, the software runs as an application on the peripheral devices 510.

High-Level Simulation Model

The high-level simulation model has two purposes: simulating the disease propagation in the university campus (students living in a certain area and have similar behavior patterns and characteristics) and provide what-if analysis for evaluation of pandemic control policy for different stakeholders (e.g. University leadership, Registrar etc.).

In an exemplary embodiment, in a COVID-19 disease propagation model of the University of Arizona (UA) campus, the dataset stores academic building information (i.e. ENGR building, 32.232793° N,110.953155° W, Classroom ENGR 301, Capacity 34 students, Size 714 sf×8.3 f), based on the interactive map website of UA (https://interactivefloorplans.arizona.edu/) Dormitory/Off-campus housing building information (i.e. Likins Hall, 32.228067° N,110.950479° W, 12 single rooms, 164 double rooms for 340 students, total capacity 369 students), based on the information provided by the campus housing department of UA Campus Health building information (i.e. 32.228131° N,110.951971° W, Open time: Mon to Fri, 8:00 am to 4:30 pm, Capacity 500 Antigen test, unlimited PCR test). Schedule of Individual Agents (i.e. Mon, 8:00-8:50, ENGR building Room 301; Mon, 9:00-9:50, Student Union; . . . ; Mon, 20:00-Tues, 7:00, Likins Hall Room 24; . . . ; Fri, 16:00-16:30, Campus Health; . . . ; Sat, 20:00-22:00, Sorority House A (if party event)), based on the class information of Stage 1 provided by register office of UA.

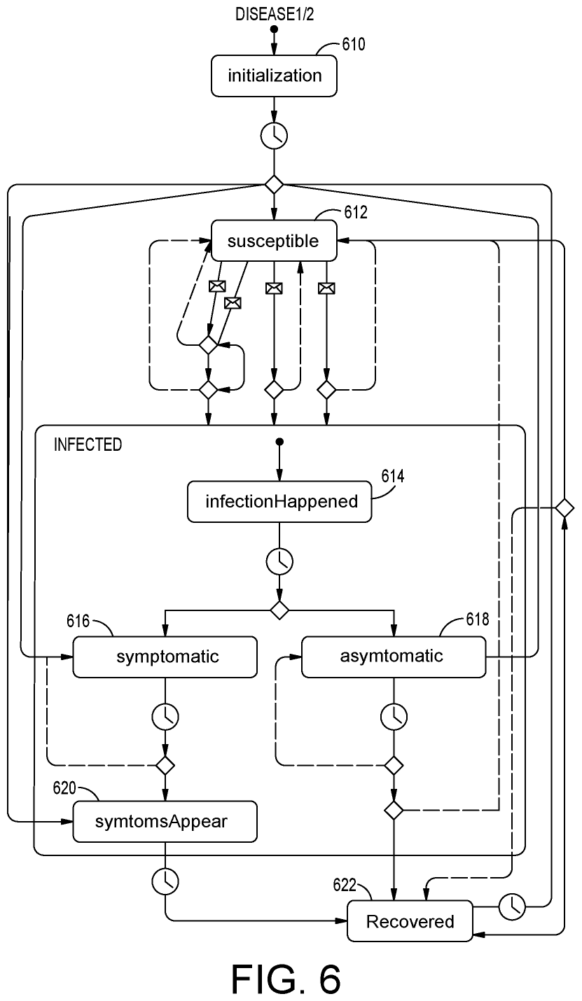

A disease propagation dataset is configured to store the different states of disease (transition between each health status) and parameters (incubation period and viral shedding amount). In an exemplary embodiment, the disease propagation dataset is described with respect to FIG. 6. The disease propagation state-chart determines the state of each data point in the set as follows: At “Initialization” 610, the dataset analysis of the disease propagation state commences. The “Susceptible State” 612, defines a state where, when agents in the Susceptible State is exposed, he or she will transit to the Pre-Symptomatic State via 0.6*p, the Asymptomatic State via 0.4*p, or stay in Susceptible State via 1−p (p is the infectious risk parameter which will be introduced in Method part). When agents enter Pre-symptomatic State, they will receive an incubation period day (range: 1 day to 20 days) via probability (i.e. 1-day incubation period, 0.00004; 2-day incubation period, 0.011842; . . . ). When agents are in 7-day before symptom onset, they will start to shed virus according to days (viral shedding rates: i.e. 1-day before onset, 10²/m³; 2-day before onset 10^{1.8}/m³; . . . ). When agents stay in this state as long as their incubation period days, they will transit to Symptom Onset State 614.

When agents enter Asymptomatic State 618, they will receive a disease period day (range: 1 day to 10 days) via probability (i.e. 1-day disease period, 0.000042; 2-day disease period, 0.012435; . . . ). And they will start to shed virus according to days (disease period day 1, 10⁻¹/m³; disease period day 2, 10⁰/m³; . . . ). When agents stay in this state as long as their disease period days, they will transit to Recover State via probability 0.5152, or transit to Susceptible State 612 via probability 0.4848.

When agents enter Symptom Onset State 620, the software will schedule a test with Campus Health and go to receive the test on the same day (if in open time) or next day (if not in open time). When agents stay in this state for 14 days, they will transit to Recover State via probability 0.8750, or transit to Susceptible State via probability 0.1250. When agents enter Recover State 622, they will have immune ability so that even exposed to the infected, they will be safe. When agents stay in this state more than 14 days, they will transit to Susceptible State according to days (14 days, 0.0873; 15 days. 0.1245, . . . ).

The Pre-symptomatic 616 and Asymptomatic State 618 are specific for COVID-19. If the model is simulating another disease such as the flu, then there will not be an Asymptomatic State. Alternately, if the model is simulating a disease like cholera, then there will be an additional Environmental Reservoir State.

Keep Social Distance: Agents will try to maintain a 6-feet social distance between each other both indoor and outdoor. Social distance violation parameter: 0/0.15/0.25 (Optimistic/Moderate/Pessimistic scenario). Wear Mask Indoor: Agents will wear a mask when they are taking classes (if they have a mask) and have other indoor activities. Mask wearing policy violation parameter: 0/0.15/0.25 (Optimistic/Moderate/Pessimistic scenario).

On-Campus Regular Test: Agents live in dormitory will receive the Antigen test per two-week. Agents who are dancers or sport team members will receive the Antigen and PCR test per two-week.

Manual Contact Tracing: Agents who have a roommate or attend a party, if one of their roommates or party attendees received test Positive results, they will stay in quarantine states for 4-5 days and schedule a test with Campus Health. Manual Contact Tracing Effectiveness: 1/0.9/0.85 (Optimistic/Moderate/Pessimistic scenario). Isolation and Quarantine: Agents who receive Positive results and in Symptom Onset State, will go to Isolation. Agents who receive Positive results and Not in Symptom Onset State, will go to Quarantine.

A disease propagation input is configured to set the disease propagation strategy, % of infected population; set the agents behaviors, % of mask wearing. Based on the input from user, it is optional to generate multiple scenarios for analysis. In an exemplary embodiment, in the COVID-19 disease propagation model of UA campus, the disease propagation input for Week 6 was: 3.21% Infected (1.86% Symptomatic, 1.35% Asymptomatic), 2.28% Isolation/Quarantine, 1.20% Recovered. 90% Mask Wearing. The preceding served as the prospective scenario. The best case scenario was 5.16% Infected (3.10% Symptomatic, 2.06% Asymptomatic), 2.28% Isolation/Quarantine, 2.45% Recovered. 90% Mask Wearing.

ID is the student agent id, the function read the agent schedule via its unique ID. P1-P10 refers to 10 time periods of day time: 8:00-8:50; 9:00-9:50; . . . 16:00-16:50. The 10-minute time interval is for routing and movement in the GIS map by Anylogic function which will use the path distance and walking speed to calculate the arrival time. Under most case, agent will arrive at the next destination earlier than the start of next period (leaving building 12 by 9:50 and arriving at building 3 by 9:58).

A classroom disease transmission function is configured to detect if there is any infectious agent presenting in the classroom and calculate the infectious risk p for other agents in Susceptible State after attending the class. The disease transmission for each agent is governed using the following state chart. The mathematical model for disease transmission is comprised of two parts, Droplet Model and Airborne Model. It is based on the Dose-Response Model.

The droplet infectious risk is defined as:

$${p}_{t}^{d r o plet} = 1 - exp [ - λ \times ( {\sum }_{i = 1}^{n} transmission {risk}_{i} ) \times Paricle Left \times ( \frac{C 1}{C 1 + C 2} \times d 1 \times T 1 + \frac{C 2}{C 1 + C 2} ) \times d 2 \times T 2 ]$$

$$transmission {risk}_{i} = Viral Shedding {Rate}_{i} \times Paricle {Left}_{i}$$

The airborne infectious risk is defined as:

$${p}^{airborne} = 1 - exp [ - Breath Rate \times Average Quanta Concentration \times T \times Particale Left ]$$

$$Average Quanta Concentration = \frac{Net emission rate \times Paricle Lef t}{R \times V} \times ( 1 - \frac{1}{R \times T} ) \times ( 1 - exp ( - R \times T ) )$$

In the exemplary COVID-19 disease propagation model of UA campus, for the Droplet formula: λ is a COVID-19 specific parameter, indicating probability that one viral particle establishes infection×conversion from arbitrary units. λ=3.78×10⁻⁶. Transmission risk is calculated on Agents in Pre-symptomatic State or Asymptomatic State side. Particle Left is the particle left with (Particle Left=0.3) or without (Particle Left=1) wearing a mask. C1 is the number of contacts in 0-3 feet of one Agent in Susceptible State. C2 is the number of contacts in 3-6 feet of one Agent in Susceptible State. d1 is the cough-droplet specific parameter indicating particle spreading in 0-3 feet space area. d2 is the cough-droplet specific parameter indicating particle spreading in 0-3 feet space area. T1 is the cumulative time period of contacts in 0-3 feet of one Agent in Susceptible State. T2 is the cumulative time period of contacts in 3-6 feet of one Agent in Susceptible State.

In the exemplary COVID-19 disease propagation model of UA campus, for the Airborne formula: Breath Rate is average breath rate for students, 0.8 m³/h. T is the class duration, 0.83 h (50 minutes) in this model. Particle Left is the particle left with (Particle Left=0.3) or without (Particle Left=1) wearing a mask. Net Emission Rate is a parameter related to particle exhaled by Agents in Pres-symptomatic State or Asymptomatic State. Net Emission Rate=16 q·h⁻¹. R is the first-order loss rate, 3.62 h⁻¹. V is the classroom volume (m³).

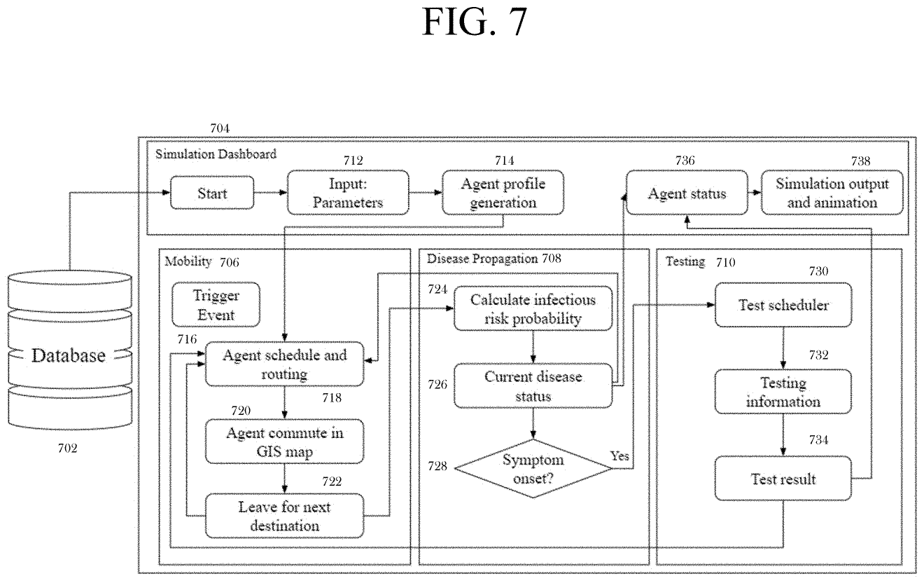

In this context, agent-based simulation has been utilized to represent behavior of students. FIG. 7 shows the system architecture with information flows within the analysis to handle agent's mobility, disease transmission, and testing. Real data has been utilized to initialize the parameters for class schedules, testing policies, dormitory capacities, and associated infectious risks. In specific, mobility component serves as the foundation of disease propagation as it defines the interaction between different human agents and the interaction between human agents and environment due to various activities during commutation through the GIS map. As shown in FIG. 7, the system is comprised of a database 702, a simulation dashboard module 704, a mobility module 706, a disease propagation module 708, and a testing module 710.

The simulation dashboard module 704 collects data from the database 702 at “Input: Parameters” 712, and that data is used by the module 704 at “Agent profile generation” 714 to create agent profiles. The mobility module 706 monitors for trigger events 716 and is comprised of modules for agent scheduling and routing 718, agent commutes (movements) in the GIS map 720, and tracking for when agents leave for their next destination 722. The agent profile generated by the dashboard 704 is transmitted to agent scheduling and routing 718, which uses agent commutes (movements) in the GIS map 720 and tracking for when agents leave for their next destination 722.

The disease propagation module 708 is comprised of modules that calculate infectious risk probability 724, the current disease status 726, and a symptom onset analyzer 728. Whenever an agent leaves for a new destination 722, the disease propagation module 708 calculates the infectious risk probability 724 and determines the agent's current disease status 726. The system also determines whether or not there has been an onset of symptoms 728. If there is an onset of symptoms, the software process moves to the testing module 710, which is comprised of the test scheduler 730, the testing information module 732, and the test result module 734.

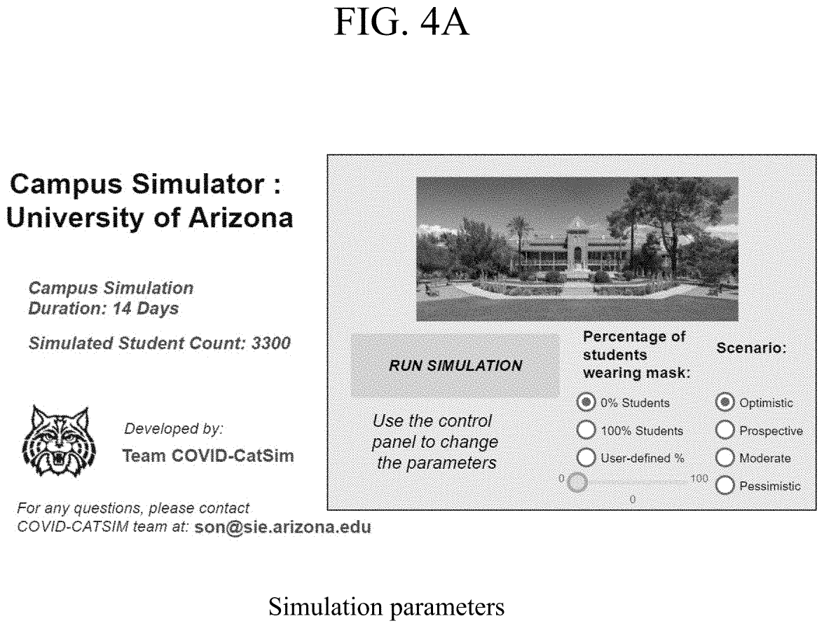

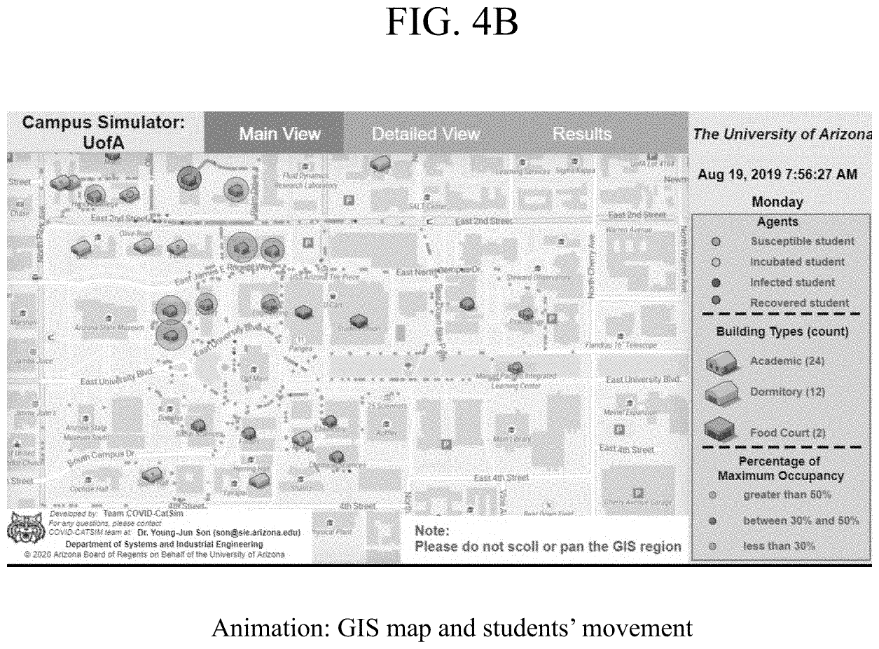

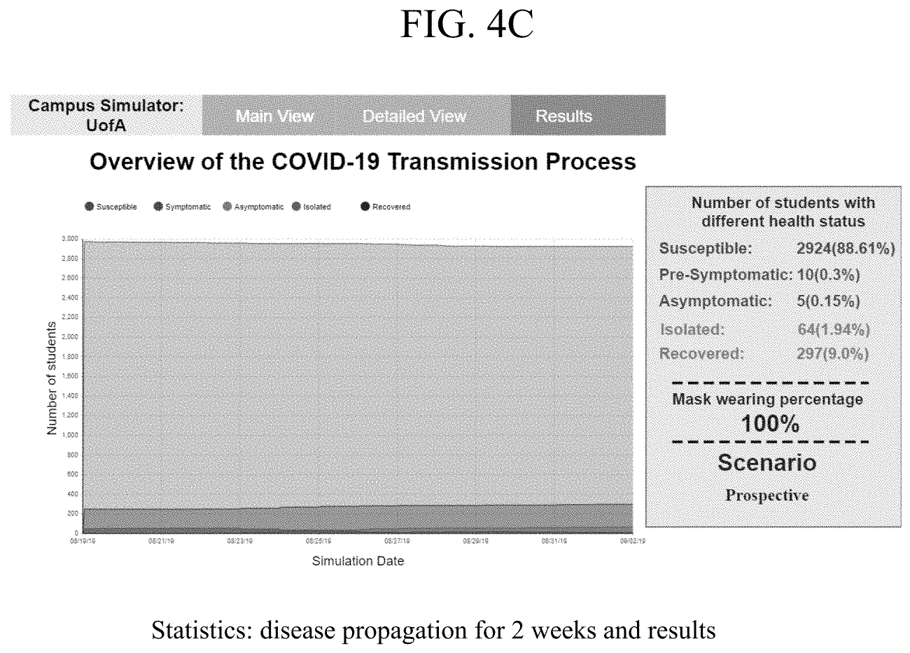

At the testing module 710, the test scheduler 730 may offer appointments or automatically schedule an appointment for testing for the agent, while the testing information module 732 will provide information related to the testing. The testing module 710 generates different false negative probability and false positive probability based on the current viral shedding rate of disease status 726 and symptom status 728 of each agent transmitted by the disease propagation module. Once testing is complete, the test result module 734 will provide results to the agent as well as to the agent scheduling and routing module 716, which will instruct software to recalculate infectious risk probabilities 724 and update the agent's current disease status 726 based on the testing result 734. That data will be transmitted to the agent status module 736 of the simulation dashboard 704, which will output the current status and locations of all agents in the simulation 738 through a graphic user interface (for example, as shown in FIG. 4B).

The Anylogic simulation model uses Dijkstra's algorithm to define human agent routing behavior simulation between origin and the destination. Moreover, the indoor space contact model was utilized to simulate the agent movement and contacts for social distance violations inside indoor facilities. The infectious risk for each agent is calculated via the disease propagation model with droplet transmission model, airborne transmission model, residential transmission model and off-campus transmission model during departure from the current indoor facility. Finally, the testing component model is introduced to simulate the test and treat policy of the university with the test accuracy model, which focuses on the false positive and false negative results formulated based on the disease propagation information. The statistical data, for example, the daily new infected, the daily test positive case, and the daily amount of agent at each disease propagation status, are synchronously updated in the simulation animation dashboard and stored in the export excel file.

Dijkstra's Algorithm

Anylogic's GIS functionality was used to mimic the agents' movement from one location to another. Based on the latitude and longitude coordinates, we defined all facilities (e.g., academic buildings, dorms, recreational facilities, and healthcare facilities) as GIS Points. Then, we created a GIS network by modeling all of the movement paths throughout the university campus as GIS routes and connecting them with GIS points. The agents in the network follow Dijkstra's shortest path algorithm to move from one building to another. The GIS network's integrated shortest path algorithm assists in determining the shortest path between places, calculating the walking time based on the agent's walking speed, and reflecting it in the simulation animation.

To identify the shortest path from the starting point u to the final vertex v, Dijkstra's algorithm assigns a distance label that specifies the shortest length from the starting point u to the other vertices s of the graph. The algorithm works in steps to reduce the value of the vertices' label at each stage. The label at the starting point u is zero (d[u]=0); however, the labels in the other vertices s are infinity (d[s]=∞), implying that the distance between the starting point u and the other vertices is initially unknown. There are n iterations in Dijkstra's algorithm. If all vertices have been visited, the algorithm ends; otherwise, the algorithm chooses the vertex with the smallest value (label) from the list of unvisited vertices (starting with u). The method then considers all of this vertex's neighbors (vertices that have common edges with the initial vertex) and calculates a new length for each unvisited neighbor, which is equal to the sum of the label's value at the initial vertex s, (d[s]), and the length of the edge e that connects them. If the resulting value is smaller than the label's value, the algorithm replaces the label's value with the newly obtained value.

$$d [ neighbors ] = min ( d [ neighbors ] , d [ s ] + e ) \qquad ( 1 )$$

After n iterations, all of the graph's vertices will be visited, and the algorithm will terminate. Then, the algorithm identifies a path array p [ ] and stores vertices in the shortest path to restore the shortest path from the beginning point to other vertices. In other words, the full path from u to v is:

$$P = ( u , … , p [ p [ p [ v ] ] ] , p [ p [ v ] ] , p [ v ] , v ) \qquad ( 2 )$$

Indoor Space Contact Model

As part of the campus re-opening approach, some colleges and universities introduced hybrid programs and reduced in-person class capacity to lessen transmission risk in the classroom. For this research, we utilized the indoor space contact model to mimic student agents' contracts, exposure, and physical distance violation behavior during the entrance and exit operations from a classroom-type indoor facility. The pedestrian dynamics in classroom-like facilities (e.g., classroom, meeting room, office room, auditorium, dance class) were modeled and analyzed using an agent-based modeling approach that took into account physical distancing, seat assignment, and entrance and exit policies. To execute multiple what-if assessments under different policies, comprehensive simulation modeling, analysis, and the amalgamation of real data including layout (e.g., traditional classroom layout, collaborative classroom layout), class schedules, seating arrangement, and permissible capacity were considered.

Droplet Transmission Model

As a main mode of COVID-19 transmission, infectious individuals tend to transfer the virus to a healthy individual via respiratory droplets when chatting, coughing, or even breathing. To simulate the progress of virus shedded from one infectious agent and inhaled by another susceptible agent, firstly, we consider the aerodynamics of respiratory droplets and the amount of pathogen-carrying aerosol droplets under which a susceptible agent will expose when he is at a certain distance. Secondly, we calculate the viral particle amount in the droplets according to the infectious agent's disease status, while considering factors such as mask wearing. Subsequently, a dose-respond model calculates the probability by the total amount of virus inhaled during the contact period for a susceptible agent in close contact with that infectious agent.

A slender Gaussian plume model is used to calculate the concentration of the droplets (C_{droplet}) in a homogenous turbulent flow at certain height of a person's face and at a certain distance (x) with the rate of emission (Q) based on the breath rate, the initial speed of droplet (U=50 m/s for sneezing, 10 m/s for couching and nm/s for breathing), and a random height difference between two individuals (ΔH). To simplify the model, we only consider the face to face interaction and set the other 2 directions as 0.

$$\begin{matrix}{C}_{droplet} ( x , 0 , 0 ) = \frac{Q}{U} \frac{1}{π {σ}_{y} {σ}_{z}} {e}^{\frac{- Δ {H}^{2}}{2 {σ}_{z}^{2}}} , & ( 3 ) \\ {σ}_{y} = {I}_{y} x , {σ}_{z} = {I}_{z} x , & ( 4 ) \\ {I}_{y} = 0.15 , {I}_{z} = 0.05 & ( 5 )\end{matrix}$$

Assuming the contact duration (T), the breath rate (R), the viral shedding rate (Rᵥᵢᵣₐₗ) and the mask efficiency (M), with the exponential dose-response curve helped in calculation of the infectious risk within close contact. Furthermore, λ represents virus-specific parameters for the probability of a single pathogen to initiate the infection.

$${p}_{droplet} = 1 - {e}^{- λ \cdot T \cdot R \cdot {R}_{viral} \cdot {C}_{droplet} \cdot M} \qquad ( 6 )$$

The model focused on contacts in two distance ranges, 0-3 feet and 3-6 feet. The contact duration in each distance range is sampled from a more detailed agent-based simulation model for indoor activities [low level model ref]at different indoor spaces based on functionality (e.g., small classroom, large classroom, meeting room, food court, auditorium, dance class) to mimic generic university wide activities. To represent realistic human movement within the indoor spaces, the indoor movement model utilized pedestrian dynamics with embedded social force and proposed a physical distancing framework that incorporated deadlock detection and resolution mechanisms. Herein, we used the distribution of individual agent's exposure duration (the average time one student spends within a specific distance of other students) and number of contacts with others (the average time one student spends within a specific distance of other students) at two different distance ranges (0-3 feet and 3-6 feet) to calculate transmission risk at different indoor places (e.g., class, offices, gatherings, party).

Airborne Transmission Model

For indoor activities, we use an aerosol transmission estimator tool developed by University of Colorado-Boulder to calculate the airborne transmission risk based on the dose-response model. In the model, we emphasized on the classroom volume (V), class duration (T), ventilation condition of the classroom (R_{V}), and the mask efficiency (M). The average quanta concentration (AQC) is a standard dynamic response of increasing aerosol quanta concentration in a room, assuming the initial quanta concentration is 0, air is well-mixed, and the infectious agent as a constant input of viral droplets. RN represents the net emission rate and, in the model, we updated it with the viral shedding rate to follow the consistency with the droplet transmission model. And C is the virus specific constant parameter considering the viral decay rate and deposition to surfaces rate based on viral particle size.

$$\begin{matrix}{p}_{airborne} = 1 - {e}^{- R \cdot AQC \cdot T \cdot M} & ( 7 ) \\ AQC = \frac{{R}_{N}}{( {R}_{V} + C ) \cdot V} \cdot ( 1 - \frac{1}{( {R}_{V} + C ) \cdot T} ) \cdot ( 1 - {e}^{- {R}_{F} \cdot T} ) & ( 8 )\end{matrix}$$

Residential Transmission Model

The residential transmission model is used to calculate the transmission probability when an agent is sharing a room with his roommate or family members. Considering the complexity of daily close contacts and the transmission via shared space, especially the restroom as a source of fecal transmission, we use the data from a cohort study focusing on the secondary transmission risk in households. Assuming the secondary transmission risk as 23% and adult secondary attack rate as 69.6%, the probability of transmission was fitted to the viral shedding rate (Rᵥᵢᵣₐₗ) with the dose-response model (k is a constant value).

$${p}_{{residential}_{total}} = {\sum }_{i = 1}^{n} ( 1 - {e}^{k \cdot {R}_{viral}} ) \qquad ( 9 )$$

Off-Campus Transmission Model

In this simulation, we consider the interaction between university affiliations with local people as an external source of new infections. The basic reproduction number, R₀, is widely used as a public health index representing the average number of secondary cases introduced by one infectious agent in a community. As shown in the Equation 10, we calculate the off-campus transmission risk with the R₀ value of the zip code zone around the university. The pᵢₙᵢₜᵢₐₗ presents for the initial infectious percentage in the zip code zone area as a simulation input. The R₀ minus the Rresidential since we consider it a separate transmission risk (R_{residential}=0.23 as shown in the previous section). N presents for the percentage of the university affiliation amount in simulation of the total population in the university zip code zone, D presents for the infectious window for the disease, and F is a control parameter between 0 and 1 related to the off-campus activity frequency controlled in the simulation.

$${p}_{off - campus} = \frac{{p}_{initial} \cdot ( {R}_{0} - {R}_{residential} \times N )}{D} \cdot F \qquad ( 10 )$$

Test Accuracy Model

In this model, as we simulate the agent at different disease status with different viral shedding rates, we also emphasize simulating test accuracy of the Antigen and PCR test. If one infectious agent, especially the asymptomatic, cannot be identified by these tests correctly, they may contact other agents and spread the disease broadly.

As a fast test method, the Antigen test has a superior performance for the symptom onset patients who have a higher viral load. Combined with the result from a meta study, Antigen test has a 78.3% (78.3-84.1%) positive rate for symptom onset patient within the first week after symptom onset, and a 51.0% (40.8-61.0%) positive rate in the second-week after symptom onset. In addition, the Antigen test shows positive result when observing the detector material conjugated with the viral particles.

For the PCR test, according to a study which repeatedly tested 200 patients in UK using PT-OCR techniques, it showed that the PCR test detected infection peaked at 77.0% (54.0-88.0%) 4 days after infection, decreasing to 50% (38-65%) by 10 days after infection. Since this analysis only focused on the positive rate along with the days since infection, we fit this result with viral shedding rate according to the discrete probability distribution of incubation period, and adjusted the probability of positive rate at each level of viral shedding amount via the viral load [39]: for low cycle threshold values (Ct<25), the positive rate is 94.5% (91.0-96.7%) and for high cycle threshold value (>25), the positive rate is medium 40.7% (31.8-50.3%).

We assume the conjugated rate has a positive relationship with viral load of the test sample, subsequently fitting the probability distribution using the Gaussian estimation for the mean x̅ and variant σ from existed research, using the viral shedding amount Rᵥᵢᵣₐₗ of different disease status as the estimation of viral load to calculate the table of test positive rate, and a sigmoid function to decide if the test result is positive or not as shown in the Equation 11.

$$p ( positive ) ∝ \frac{1}{1 + {e}^{F ( f ( {R}_{viral} ) , \overline{x} , σ )}} \qquad ( 11 )$$

Model Configuration—Mobility

In the mobility component, two types of inputs include: the building agents and the time-location-activity schedule of human agents. For the building agents in the campus simulation, there are multiple categories, such as the academic buildings for attending the classes, the dormitory to provide accommodation, the food court to account for interactions during meals, leisure space to conduct different activities with agent groups, the test centers to get tested, and the isolation dormitories to isolate infected agents and give treatment. For each building agent, it requires several parameters: GIS information for routing algorithm calculation, the physical size of indoor space, ventilation rate for airborne transmission calculation, building capacity for occupancy visualization on the GIS map, and it also stores the disease propagation information of human agents who present at this location.

The agent's schedule is comprised of durations, location and agent activity (see Table 1). Each agent contains its schedule in the simulation, and every daily schedule is different during a week. In the current model, the basic time unit is one hour. Therefore, at the beginning of each hour, the model will read the activity and destination of this hour, locate the destination on the GIS map, calculate the route and make a movement with the agent's walk speed if necessary, and set the activity as a component of agent's current status (see Table 2).

TABLE 1
The example of agent schedule for Monday

| | | Time | Building | Building | | |
|---|---|---|---|---|---|---|
| ID | Type | period | ID | Name | Room | Activity |
| 1 | Student | 8:00-8:50 | 4 | Honor Village | 203 | Rest |
| 2 | Student | 8:00-8:50 | 4 | Honor Village | 210 | Ling 213 |
| | | | | | | (Online) |
| 3 | Faculty | 8:00-8:50 | 72 | Engineering | 302 | Engr 310 |
| | | | | | | (In-person) |
| 4 | Student | 8:00-8:50 | 72 | Engineering | 302 | Engr 310 |
| | | | | | | (In-person) |
| 5 | Employee | 8:00-8:50 | 100 | Bursar | 100 | In work |
| | | | | Building | | |

TABLE 2
The algorithm of mobility component

```
Input:
t = simulation time
lᵢ(t) = location of human agent i at time t
(Lᵢ(T), Aᵢ(T) = human agent (student, faculty, and staff) i′s schedule containing agent's location, activity
        and is a function of time T. T is the pre-defined schedule time (HH: mm, Month, Day)
L_{b}(t) = building agents containing the list of building locations and their corresponding information
        (e. g., occupancy, number of infected student in the building) which updates based on time t
Dᵢ(t) = disease propagation status (susceptible, symptomatic/asymptomatic, recovered, and
        corresponding parameter values) of human agent i at time t
A_{d} = array of essential activities (e. g. , lecture, mandatory test, break − time, groceries)
A_{c} = array of flexible activities (e. g. , gathering, party, long − weekend mobility)
nᵢ = number of participants in human agent i's activity
Nₖ = total number of human agents participating in activity k ⊂ Ac
Procedure:
when t == T
        if lᵢ(t) ! = (Isolation Dormitory | Self − quarantine) then
        if Aᵢ(t) ⊂ A_{d} then
              move agent i to Lᵢ(T)
              add Dᵢ(t) to L_{b}(t) for L_{b}(t) == Lᵢ(T)
        else
              Aᵢ(t) = k ⊂ Ac ← p(A_{c} )
              nᵢ ← f(Nₖ, p (Aᵢ(t))
              move agent i to L(Aᵢ(t))
              add Dᵢ(t)to L_{b}(t) = L(Aᵢ(t)) for Lb(t) == Lᵢ(Aᵢ(t))
        calculate (CNᵢ, DTᵢ)
        if positive test result ← Dᵢ(t)
              if agent i ⊂ on − campus population
                    move agent i to Isolation Dormitory
              if agent i ⊂ off − campus population
                    move agent i to Self − Quarantine
Output:
L_{b}(t) = building agent containing the list of building locations and their corresponding information
(e. g. , occupancy, number of infected student present on the building) which updates based on time t
(CNᵢ, DTᵢ) = contact number and contact duration of agent i per contact distance 0-3 feet and 3-6 feet
```

Model Configuration—Disease Propagation

The disease propagation part mainly has two components: the disease status of agent (viral shedding rate for infected status, immune period for recovered stated) and the infectious probability calculation.

For the disease status component, the COVID-19 simulation case includes the susceptible state, the infected state, and the recover state. And the expose state is considered as an integration of mobility part and infectious risk calculation component. When initializing the simulation, every agent will be assigned a disease status according to the user input. When exposed to an infectious agent, the model will calculate the infectious risk as the transition probability to infectious state for the agent in a susceptible state. Once the agent enters the infectious state, firstly, it will be categorized as a symptomatic infectious agent or an asymptomatic agent for most infectious disease simulations. And it will be assigned an incubation period according to a probability distribution, and the model will read the viral shedding rate via the day before symptom onset. Specifically, for the asymptomatic agent, the ‘symptom onset day’ is considered as the peak of viral shedding rate (see Table 4). After the infectious agent receives the treatment or recovered by himself, it will transit to recover state with the disease severity based immune window and probability. When the agent reaches the last day of the immune window, it will transit to susceptible state. Specifically, considering the vaccination, when a susceptible agent is vaccinated (received the second dose), it will transit to the immune state after 14 days. The effectiveness of different vaccines could be considered the transition probability.

The infectious probability, as introduced in the previous section, is based on both location and activity. At the beginning of each time period window, as human agents arrive at their destination, they interact with building agents and the building agents aggregate the disease information for each room, as the amount of infectious agent (with viral shedding rate), the amount of susceptible agent, and the amount of recovered agent. Then the building agents interact with transmission models per building type and activity type (see Table 3) to calculate the infectious probability for the susceptible agent as shown in Table 4.

TABLE 3
The interaction between building agent, model and infectious risk calculation

| Building Agent Type | Activity Type | Human Agent Type | Interacted Model |
|---|---|---|---|
| Academic Building | In-person Class | Faculty, Student | Droplet*, Airborne**, Indoor*** |
| | Office/Lab work | Faculty, Student | Airborne |
| Administration Building | Office work | Employee | Airborne |
| Service Building | Service | Employee, Student | Droplet, Airborne |
| In-campus Dormitory | Rest/Online Class | Student | Residential**** |
| Off-campus Dormitory | Rest/Online Class | Faculty, Employee, Student | Residential, Off-campus***** |
| | Party | Faculty, Employee, Student | Droplet, Airborne |
| Leisure Space (Indoor) | Gathering | Student | Droplet, Airborne |
| Leisure Space (Outdoor) | Gathering | Student | Droplet |
| Pub | Party | Student, Faculty, Employee | Droplet, Airborne. Indoor |

*Droplet transmission model,
**Airborne transmission model,
***Indoor Space contact model
****Residential transmission model,
*****Off-campus transmission model

TABLE 4
The algorithm of disease propagation component

```
Input:
t = simulation time
L_{b}(t) = array of building agents and their corresponding information (e. g., occupancy, number of infected
        student present on the building) which updates based on time t
(CNi, DTi) = contact number and contact duration of human agent i per contact distancerange
Dᵢ(t) = disease propagation status of agent i at time t
rₛ = percentage of asymptomatic disease group within student agents
Sym(day, probability) = incubation period length based on discrete probability distribution
Asym(day, probability) = infectious period length based on discrete probability distribution
T_{incubation} = incubation period for human agents in symptomatic state
T_{infectious} = infectious window period for human agents in asymptomatic state
T_{recover} = recover period for human agents in symptomatic state
Tᵢₘₘᵤₙₑ = immune period for human agents in recovered state
tᵢ(0) = first day (time) of infection for human agent i
Vᵣ(t) = viral shedding rates per day prior to symptoms onset
Procedure:
when t == T
        for susceptible ∈ Dᵢ
                if rand (0,1) < f(L_{b}(t), ((CNᵢ, DTᵢ)))
                        update Dᵢ ← (Symptomatic, Asymptomatic) ←p (Symptomatic, Asymptomatic)
        for Symptomatic ∈ Dᵢ
                T_{incubation}, T_{recover} ← Sym(day, probability), tᵢ(0) = t
                while t − tᵢ(0) < T_{incubation}
                        update Dᵢ ← Vᵣ(T_{incubation} − (t − tᵢ(0)))
                while t − tᵢ(0) > T_{incubation} & t − tᵢ(0) < T_{incubation} + T_{recover}
                        Dᵢ ← symptom onset, Vᵣ(symptom onset)
                while t − tᵢ(0) > T_{incubation} + T_{recover}
                        Dᵢ ← recover;
                        Tᵢₘₘᵤₙₑ = f(max(Vᵣ))
                while t − tᵢ(0) > T_{incubation} + T_{recover} + Tᵢₘₘᵤₙₑ
                        Dᵢ ← susceptible
        for Asymptomatic ∈ Dᵢ
                T_{infectious} ← Asym(day, probability), tᵢ(0) = t
                while t − tᵢ(0) < T_{infectious}
                        update Dᵢ ← Vᵣ(T_{infectious} − (t − tᵢ(0)))
                while t − tᵢ(0) > T_{infectious}
                        Dᵢ ← recovery;
                        Tᵢₘₘᵤₙₑ = f(max(Vᵣ))
                while t − tᵢ(0) > T_{infectious} + T_{recover} + Tᵢₘₘᵤₙₑ
                        Dᵢ ← susceptible
Output:
Dᵢ(t) = disease propagation status of human agent i at time t
```

Model Configuration—Testing

The test part cooperates with the mobility part as human agents move to campus health or test center to receive the test. The disease propagation part uses the viral shedding rate to decide the test result with test accuracy model. The time consumption of test is simulated, such as the Antigen test takes 1 to 2 hours and the PCR test takes 1 to 2 days. To simulate the real-world scenario, we set the test capacity for test facilities, for example, the daily process limitation for Antigen test is around 1000 to 1200 and for typical PCR test is 200 to 400.

The testing result is also a part of agent status and it could cooperate with other parts as shown in Table 5. For instance, if there is a manual contact tracing policy, the test positive status will be transited to building agents (academic building, dormitory) and then change the status of other human agents to quarantine.

TABLE 5
The algorithm of testing component

```
Input:
t = simulation time
Dᵢ (t) = disease propagation status of human agent i at time t
Cᵢ = binary variable representing contact tracing requirement for human agent i
    (i. e., 1 represents contact tracing is initiated and 0 represent no action)
test ᵢ = list of mandatory test schedule day of human agent i which is a subset of (Li(T), Ai(T))
testₚ = test policy (e. g., mandatory test per week, mandatory test per two weeks, voluntary test)
ttᵢ = test type for human agent i
willᵢ = testing willingness of human agent i (i.e., for voluntary test willing is generated ranging
      between (0 to1), for mandatory tests willi = 1)
Gᵢ = contacted group of human agent i defined by contact tracing policy
    (e.g., roommate, party member, classmate)
p(PCR) = probability of having a PCR test based on real world data
f _{antigen} = viral shedding rate − based function for Antigen test positive result
f_{PCR} = viral shedding rate − based function for PCR test positive result
k = parameter between (0 to1), for mandatory test policy, k between(0.8,1)
Procedure:
Initialize willᵢ
if (t in test ᵢ |Dᵢ == symptoms onset | Cᵢ == 1) && ( t == weekday)
        if rand(0,1) > willᵢ
                add t + 1 to test ᵢ
        else
                add Antigen to ttᵢ
                add PCR to ttᵢ ← p(PCR)
        for Antigen in ttᵢ, delay for rand (2,4) hour
                if f _{antigen} (Dᵢ(Vᵣ)) threshold(Antigen),
                        Dᵢ ← positive test result
                        add PCR to ttᵢ
                        for agent j ⊂ Gᵢ
                                Cj = 1
                else
                        Dᵢ ← negative test result
                        if symptoms onset ← Dᵢ
                                add PCR to ttᵢ
                                willᵢ = willᵢ * k
        for PCR in ttᵢ, delay for rand (24,48) hour
                if f _{PCR}(Dᵢ(Vᵣ)) > threshold(PCR)
                        Dᵢ ← positive test result
                        for agent j ⊂ Gᵢ
                                Cj = 1
                else
                        Dᵢ ← negative test result
Output:
Cᵢ = contact tracing condition for human agent i in contacted group
Dᵢ (test result) = test result for human agent i
```

Model Configuration—Agent Status and Behavior Rule

As mentioned above, the status of each agent in the simulation has several sub-components: location (GIS information), activity, disease propagation state, test schedule state, test result state, and other user-defined states. The agent behavior is restricted by agent status and will change the agent status. There are multiple status restriction rules using in the current simulation model, such as the isolation/self-quarantine state (no new movement allowed), the vaccinated state (test exemption), and the rest state (gathering activity only select agents who are in the rest state, not working or taking classes). And as shown in the test part, if an agent goes to the test and gets a positive result, he will change agents in his contact group to be in-contact-tracing status.

Simulation and Validation—What-if Analysis

The simulation model serves a system to perform what-if analysis to help stakeholders to evaluate the impact of different policies under different pandemic stages and campus re-open stages for informed decision making. Thus, this analysis has considered what-if analysis pertaining to three factors: mask-wearing, test policy, and vaccination.

Firstly, there are several control variables introduced in the what-if analysis. The re-open condition describes how many students and employees are currently active in the campus and the capacity limitation of the in-person classes. We considered three levels of the re-open condition as stage 1 (3000 students, 3000 employees and faculty and staff, with classroom limitation of 15), stage 2 (3000 students, 3000 employees and faculty and staff, with classroom limitation of 50), and stage 3 (6000 students, 6000 employees and faculty and staff, with classroom limitation of 50). Both initial infected percentage (0.5%, 5%) and initial immune percentage (0%, 10%, 50%) are considered and combined to simulate different stages of pandemic. The default mask-wearing percentage is set as 90%, the default test policy is set to be mandatory test per two weeks, and the default vaccination percentage is set to be 0%.

Simulation and Validation—Mask Wearing Percentage

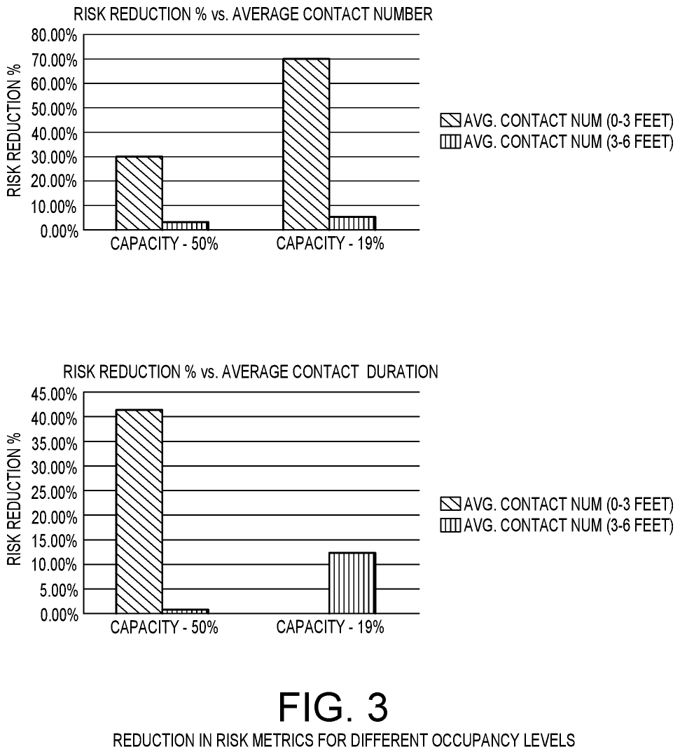

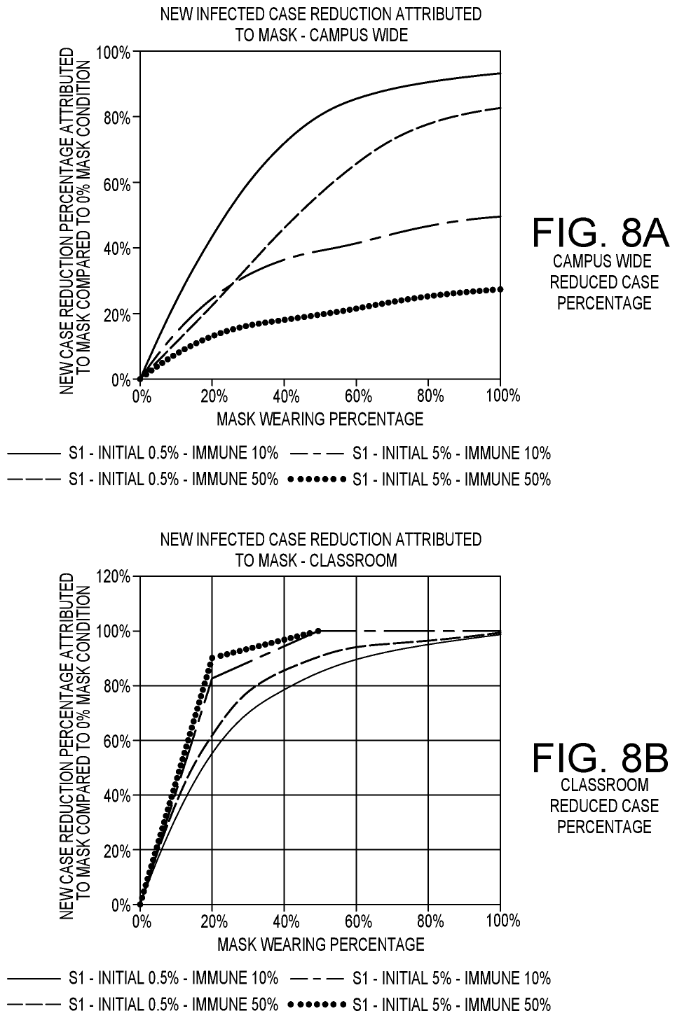

This section illustrates the importance of mask-wearing percentage and its interaction with immune percentage and campus re-open stage. FIG. 3 shows the reduced new infected cases percentage (compared with 0% mask condition) under different mask-wearing percentages and compares the reduced case percentage for classroom and campus-wide transmission. And the control variables are set to be campus re-open stage 1 (3000 students, 3000 employees and faculty and staff, with classroom limitation of 15), initial infected percentage (0.5%, 5%) and immune percentage (10% and 50%). As shown in FIG. 8A, there is a significant main effect of initial infected percentage (F(1, 16)=521.527, p<0.01), and the significant interaction effect between initial infected percentage and mask-wearing percentage (F(3,16)=17.384, p<0.05) shows that at least 80% mask-wearing percentage is essential to reduce the disease transmission risk significantly during the middle of pandemic and it could stop 79% to 94% of secondary transmission and get the situation back to control. And FIG. 8B shows that wearing a mask in extremely important for in-person class and if everyone wears the mask in the classroom, the infectious risk would be approximately to 0% even at the middle of pandemic. In addition, the counterintuitive result in FIG. 8B is that with the increasing immune or vaccinated percentage, wearing a mask for those who have not been vaccinated is the best way to protect themselves.

Simulation and Validation—Test Policy

In the test policy part, three types of test policy are analyzed: voluntary test, mandatory test per two weeks and mandatory test per one week. These tests are meant for people without any symptoms, and for people having symptoms such as cough and fever will receive both antigen test and PCR test in the campus health or campus hospital. For voluntary test, we used a gamma distribution to sample the test frequency preference for agents. For mandatory test, every agent needs to receive a test during a 7-day or 14-day test window. And for off-campus agents, the test day is associated with the day when they visit the campus.

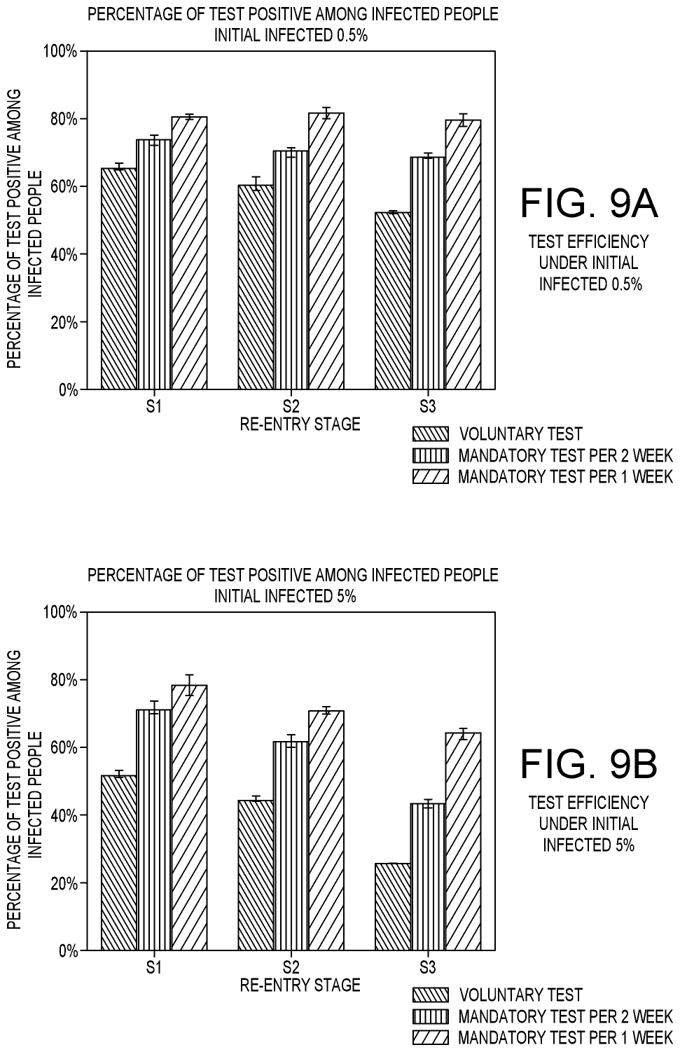

As shown in FIG. 9A, for the 0.5% initial infected scenario, there is a main effect of test policy (F(2, 18)=25,739, p<0.01), however, the difference for mandatory test per two weeks and mandatory test per one week is not significant (F(2, 12)=1.462, p>0.05). It suggests that for the low-risk periods with a small infectious rate, mandatory test per two weeks would be enough to figure out around 72% active cases by conducting a test to maintain the R₀ value less than 1. For the 5% initial infected scenario, FIG. 9B shows that applying a more strict test policy is very important as the test per two week policy for S3 is only able to figure out less than 50% and the interaction effect between test policy and re-open stage is significant (F(4, 18)=83.687, p<0.01). It suggests that mandatory test per one week is necessary to keep the campus disease propagation under control for high-risk periods with high infectious rates.

Furthermore, we selected two scenarios to compare with similar scenarios under different periods of pandemic at the University of Arizona. By September 2020, the beginning of fall semester, because of the limited capability of test material and limited in-person class, the university encouraged students and faculty members to receive the test voluntarily and most of the test are Antigen test. And by January 2021, the University of Arizona developed its own test methods, the Saline gargle test and it supported a larger test capacity which allows every university affiliation to test every week.

For the voluntary test scenario, the simulation results show that it is able to detect 13.22% cases before symptom onset, detect 9.30% asymptomatic agents with a positive result among all symptomatic cases and the estimated R₀ is 1.59. The test positive rate (positive case among total test case) is estimated to be 12.82% while the real-world scenario was 10.11%. For the mandatory test per one week scenario, the simulation results show that it is able to detect 52.80% cases before symptom onset, detect 37.83% asymptomatic agents with a positive result among all symptomatic cases and the estimated R₀ is 0.86. The test positive rate is estimated to be 2.96% while the real-world test positive rate was 2.47%.

Simulation and Validation—Vaccination Rate

In the vaccination rate part, to simplify the scenario, we considered the vaccinated population to be fully immune regardless of the mRNA vaccine (Pfizer and Moderna) efficacy as ˜82% for one dose and ˜94% for two doses. And as the infected time, location and resource of infection are traced in the simulation, we simply calculate the R₀ value based on the secondary transmission rate combining with the potential infection to external community during the off-campus activity. The mask wearing percentage is set to be 90% and the test policy is voluntary test.

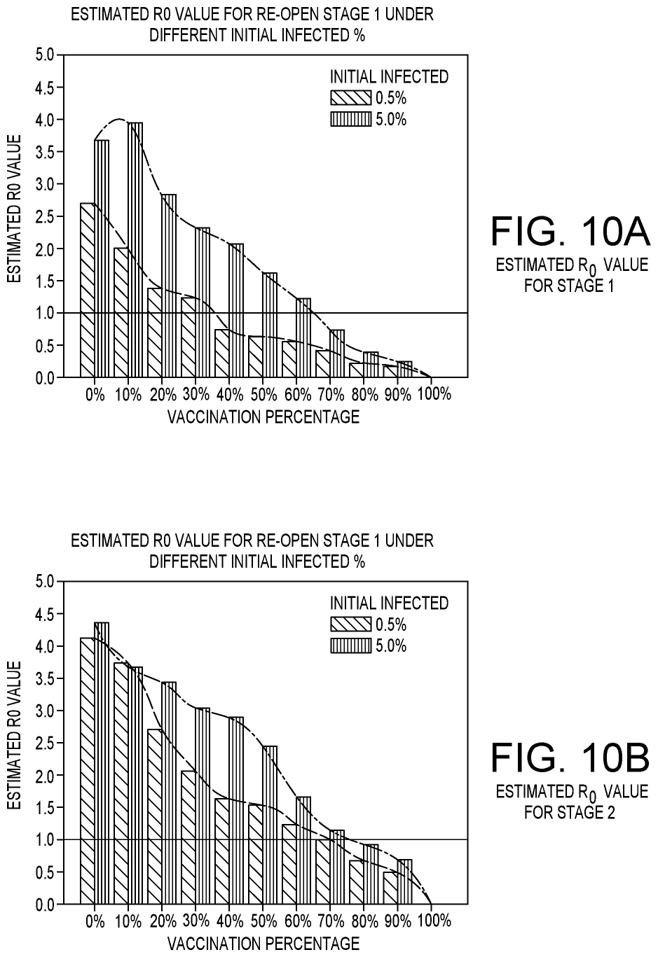

FIGS. 10A and 10B show the estimated R₀ value of different vaccination percentage regardless of those who recovered from COVID-19 with immune ability but not vaccinated. It indicates that for re-open stage 1 (FIG. 10A), when classroom capacity and social activity are constraint strictly, the disease propagation would be under control with a vaccination rate of 40% under 0.5% initial infected case and a vaccination rate of 70% under 5% initial infectious case. And for the re-open stage 2 (FIG. 10B) suggests that 70% to 80% vaccination rate is necessary to minimize the disease transmission risk. There is a significant interaction effect between vaccination percentage and initial infected percentage (F (11, 43)=4.826, p<0.01) as with a higher vaccination rate, the severe condition will be controlled sooner. And there is a significant interaction effect between vaccination percentage and re-open stage (F(11,43)=3.363, p<0.05), which suggests that with a low vaccination percentage, the university needs to be cautious to re-open the campus. Regarding the current infected percentage, the university should apply a strict test policy and minimize the group gathering event in the campus.

Simulation and Validation—University of Arizona Campus Stimulation

In this section, the simulation model is validated with two seven-week period test data from the University of Arizona main campus as the team works closely with the campus health center of the University of Arizona, providing a two-week test capacity and positive rate prediction each week. Each week's prediction will combine the policy change, vaccination rate, and student activity change to set the detailed scenario. For the student agent set, we combined the information from university residential department for the living-on-campus students (dormitory occupancy and occupancy of different types of rooms) and the hourly Wi-Fi occupancy data from university IT department to estimate the active agent in the main campus each day. For the class schedule setting, we used the information from the registrar office to set the students who enrolled in-person classes, flex in-person classes and online classes of every college and department, and related it with academic buildings and Wi-Fi occupancy data.

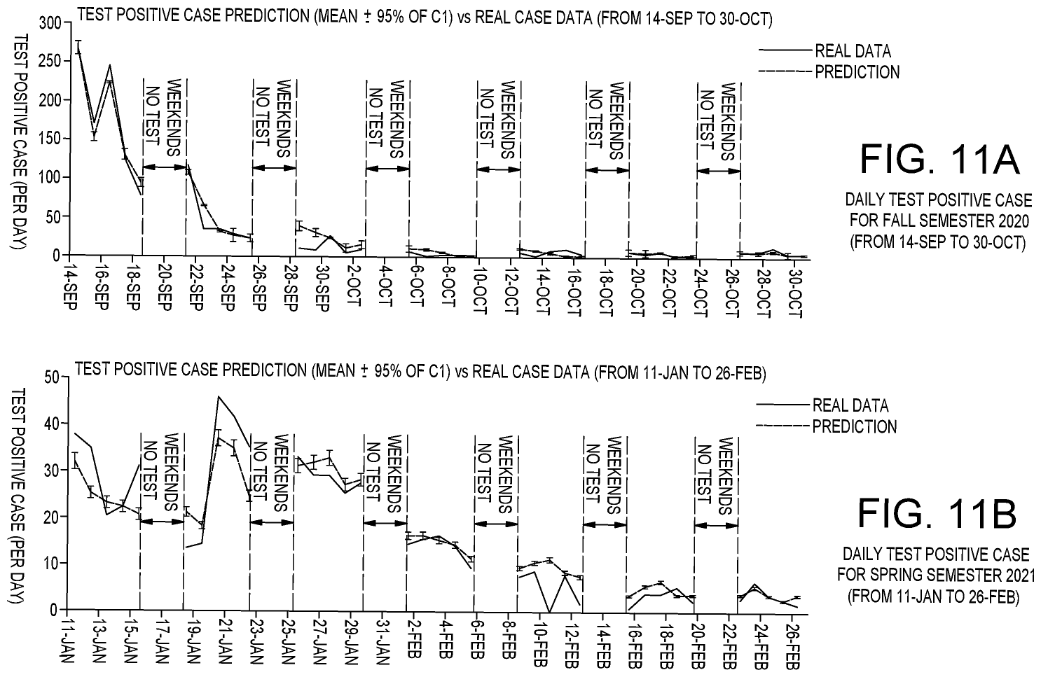

As shown in FIG. 11A, the average prediction error is 6.08 (17.25%) cases per day. For the first two weeks, as the number of test positive cases is high, the university and the government applied the lockdown policy to encourage students to stay in their dorms and apartments, avoiding gathering or parties. And the university limited the in-person class capacity to be under 30 students, requested university community to wear the mask both indoor and outdoor, and maintained a social distance as of 6 feet. However, only students who live on campus will be requested to take a test every two weeks.

As shown in FIG. 11B, the average prediction error is 2.75 (11.45%) cases per day. The university requested students to self-quarantine for seven days after travelling and taking the test in the first two weeks. And every university affiliation who has been to campus should receive a mandatory test each week to maintain their campus Wi-Fi connection. In addition, university facility department collaborated closely with university police department to minimize risky gathering and parties near the campus during the week.

The above analysis presents an agent-based campus-wide disease propagation simulation model that could be utilized as a tool for policy analysis and prediction. The model focused on the three critical factors that affect the university's re-opening stage: the current infectious case, student engagement and interaction, and vaccination status. In this analysis, we were able to replicate university affiliation behaviors of students and employees in a more organized and detailed manner using the agent-based model, estimating the infectious rate based on every agent's interaction rather than group likelihood. In addition to the internal disease propagation cycle of the university community (e.g., cohorts, roommates), the external infectious risk based on zip code specific R₀ value was taken into account since this university community also interacts with the local population, which is difficult to trace but can have a significant impact on campus transmission. The test component enabled comparison of the model results with real-world data. The infectious agent's viral shedding rate and the test type showed the percentage of pre-symptomatic and asymptomatic agents that were tested positive. In contrast, some symptomatic agent test results were false negative due to test accuracy according to test methods and viral shedding rate on the day.

Conversely, highly detailed agent behavior models have been shown to compromise long-term prediction performance in some situations. The average prediction accuracy for the first week, for example, was 93.01 percent, 88.24 percent for the second week, 76.32 percent for the third week, and 54.35 percent for the fourth week. While well-developed mathematical models were used to simulate droplet transmission and airborne transmission which are considered as major ways of COVID-19 transmission, the transmission through surface and body contact was not incorporated into the model. Although we utilized a GIS map to simulate agent movement around buildings, we did not address the pathogenic risk that arises when a 6-feet social distance cannot be maintained when the corridor is congested.

Considering the wide extensibility of this analysis, we intend to incorporate the student risk preference to simulate their route choice decision, predict the potential risk of student contact in the pathways, and provide suggestions to stakeholders for arranging some one-way pathways related to building gate position and agent flow. Additionally, this analysis can be used in campus-like modeling contexts such as K-12 education organizations, military camps, and central business districts. It is also conceivable to simulate other diseases, and seasonal flu. Furthermore, integration of the Wi-Fi data can facilitate real-time risk evaluations to show the potential risk and crowdedness of buildings throughout the campus. This agent-based campus-wide disease transmission simulation model showed a high accuracy prediction rate during the COVID-19 pandemic. It assists stakeholders in conducting what-if analysis for various policies, and also collaborates with the campus health center to anticipate test load and disease propagation situation throughout the campus in order to maximize medical resources utilization and take necessary actions.

### Example 1

In ENGR Room 301, Size 714 sf×8.3 f, Mon, 8:00-8:50, 4 agents are in this classroom taking a class. By the end of class, at the time point agents are leaving, infectious risk are calculated. Agent A in Pre-symptomatic State, 1 day before symptom onset (viral shedding rate 10²/m³), not wearing a mask (Particle left=1). Agent B in Susceptible State, wearing a mask (Particle left=0.3), have contacted in 0-3 feet (d1=0.243) with agent A, C (C1=2), the cumulative contact time in 0-3 feet is 4 minutes (T1=4); have contacted in 3-6 feet (d2=0.081) with agent A, C, D (C2=3), the cumulative contact time in 3-6 feet is 6 minutes (T2=6).

The Droplet infectious risk for Agent B is:

$${p}_{Agent B}^{d r o plet} = 1 - exp [ - 3.78 \times 1 {0}^{- 6} \times ( 1 {0}^{2} \times 1 ) \times 0.3 \times ( \frac{2}{2 + 3} \times 0.243 \times 4 + \frac{3}{2 + 3} \times 0.081 \times 6 ) ] = 0.0001$$

The Airborne infectious risk for Agent B is:

$${p}_{Agent B}^{airb o r n e} = 1 - exp [ - 0. 8 \times ( \frac{16 \times 1}{3.62 \times 167.81} \times ( 1 - \frac{1}{3.62 \times 0.83} ) \times ( 1 - exp ( - 3.62 \times 0.83 ) ) ) \times 0.83 \times 0.3 ] = 0.0033$$

The total infectious risk p for Agent B is:

$${p}_{Agent B} = {p}_{Agent B}^{droplet} + {p}_{Agent B}^{airborne} = 0.0 \; 001 + 0 .0033 = 0.0034$$

Therefore, after attending the class, Agent B will have probability 0.4*0.0034 of transition from Susceptible State to Pre-symptomatic State, probability 0.6*0.0034 of transition from Susceptible State to Asymptomatic State, probability (1−0.0034) of staying in Susceptible State.

A dormitory disease transmission function is configured to detect if there is any infectious agent presenting in the dormitory room and calculate the infectious risk p for other agents in Susceptible State after spending one night in the room. It is based on the cohort study of disease secondary transmission in family or other living space. In an exemplary embodiment of the COVID-19 disease propagation model of UA campus, the cohort study is COVID-19 specific. If roommate agent in Pre-symptomatic State or Asymptomatic, the infectious risk p for the other roommate agents in Susceptible State is generated according to his viral shedding rate (10²/m³, 0.0704; 10¹⁻⁸/m³, 0.0450; . . . ; 0/m³, 0.0000).

An off-campus disease transmission function is configured to estimate the infectious risk p based on local disease propagation condition and activity level after agents going back home and spending one night in their off-campus housing building, where:

$$p = Local Infectious Rate \times Activity Level$$

In the exemplary model of COVID-19 disease propagation on UA campus, Local Infectious Rate is estimated from daily Arizona Infectious Risk, p(24/09/2020)=0.000518, p(25/09/2020)=0.000514, etc. It is specific for every simulation date. The Activity Level is related by Shelter at Home Policy, 0.4/0.6/0.8 (Optimistic/Moderate/Pessimistic Scenario).

A party event and disease transmission function is configured to estimate the infectious risk p based on the party size and party attendee's disease status. The party is triggered by events.

$$p = \frac{Party size}{Safe group size} \times \frac{\# of Attendees in Presymptomatic / Asymptomtic state}{Party size} \times μ$$

In the exemplary COVID-19 disease propagation model of UA campus, Party size indicates the total number of agents attending this one single party. Safe group size indicates the group gathering size under permission, where Safe group size=5. μ is the COVID-19 specific party-infectious related parameter, where μ=0.5.

An isolation and quarantine function is configured to remove agents from their daily schedule and relocate them in isolation or quarantine room for a certain time period. And after that, move agents back to their daily schedule and routing. In the exemplary COVID-19 disease propagation model of UA campus: If Isolation and Quarantine Policy applied, and if agents receive Positive results and in Symptom Onset State, they will move to Isolation Dorms (in mobility part/GIS map) and isolate for 14 days. Isolate means that agents will stay in the isolation dorm and will not attend any class, party or contact with other agents. If agents receive Positive results and in Pre-symptomatic or Asymptomatic State, they will move to their Dormitory or Off-campus Housing (in mobility part/GIS map) and quarantine for 14 days. Quarantine means that agents will keep stay in the dormitory or off-campus housing and will not attend any class, party or contact with other agents.

Lower-Level Model

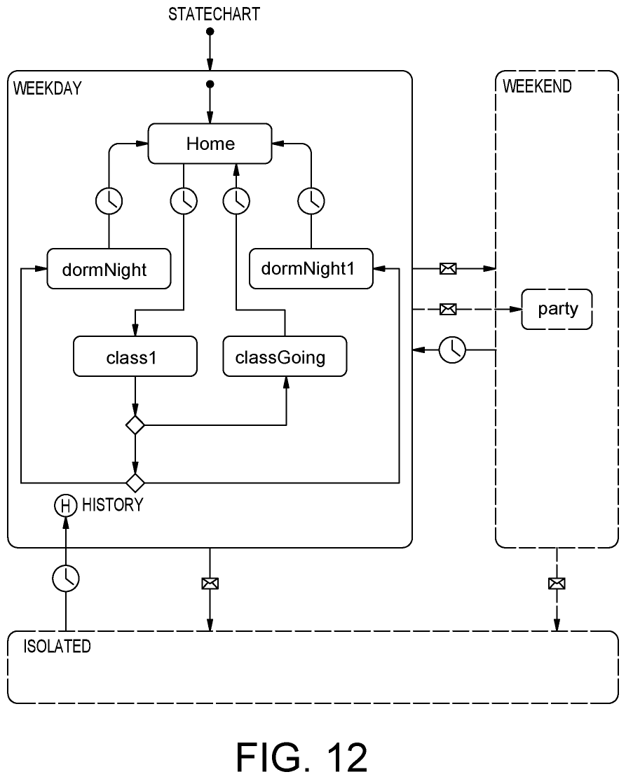

Model Input: The Model Input is shown in FIG. 12 and comprises the below.

Facility parameters under consideration
a. Capacity (maximum classroom capacity in regular operating condition)
b. Organization policy (maximum allowable occupancy, e.g. 50% of capacity, 50 people in total)
c. Entry/Exit door configurations (e.g., single door entry/exit, multiple door entry/exit)
d. Layout (e.g. Area, seating arrangement etc.)

Agent Parameter Settings
a. Speed (comfortable walking speed for human)
b. Diameter (to maintain desired physical distance)

Agent Generation
a. Arrival schedule (based on predetermined class schedule)
b. Arrival rate (based on class attendance size and class schedule)

Methodology: The Methods of the model are shown in FIG. 12 and comprise the below.

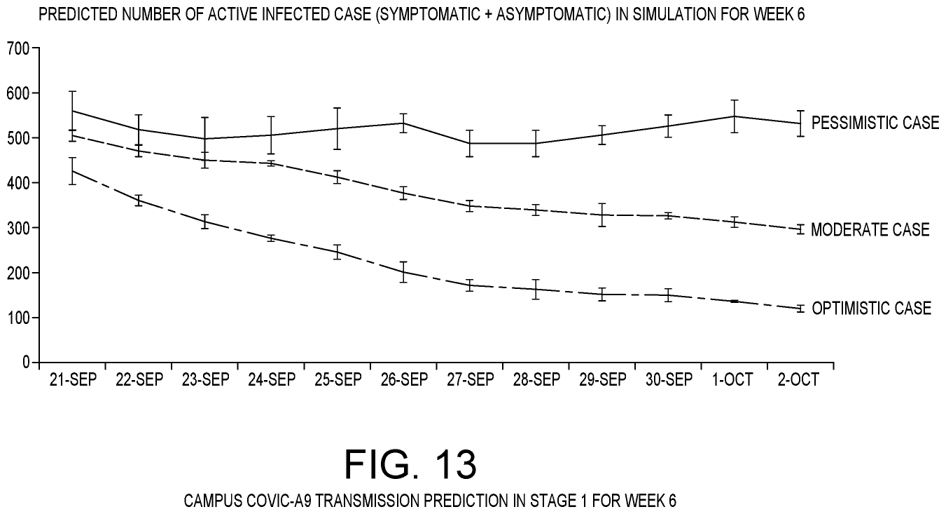

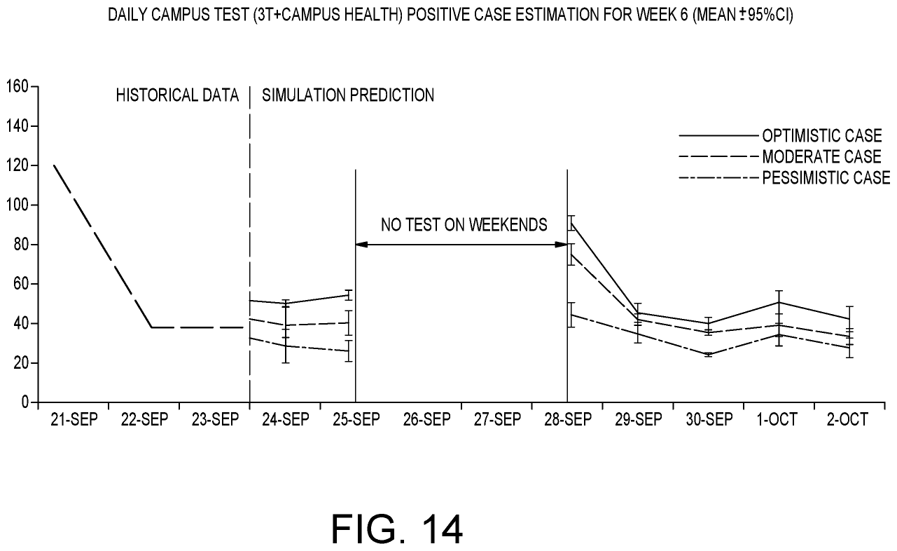

The present invention utilizes the Agent-based simulation technique in order to capture the individual agent interaction as well as evaluation of the exposure risks caused due to these interactions. Anylogic 8.5.1 has been utilized to model and implement individual agent behaviors and their interactions within the shared environment. FIG. 13 is a graph of exemplary campus COVID-19 transmission predictions for a given week based on the predictive modeling performed by an exemplary embodiment of the invention. FIG. 14 is a graph estimating the Positive state for a given week based on the predictive modeling performed by an exemplary embodiment of the invention.

Mobility:

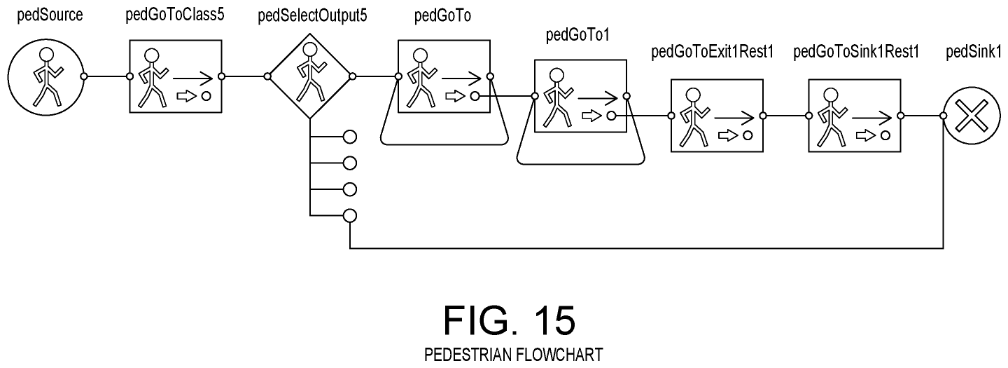

Pedestrian Library—The system has utilized the built-in pedestrian library within the Anylogic. Pedestrian library is used to assess the capacity and throughput, identify the pedestrian bottlenecks, and perform the planning within a public environment. It helps in accurate modelling, visualization, and analysis of the crowd behavior to eliminate potential abnormalities. The movement of the pedestrians using pedestrian library happen according to the social force model. Each individual agent uses the shortest path, avoids collisions with other objects as well as pedestrians within the modelled environment. The pedestrian behavior is defined using the process flowchart, which helps in gaining the understanding of pedestrian movements across the space (FIG. 15). The physical environment is comprised of the space markup elements such as walls, service points, attractors etc., (FIG. 16).

In pedestrian flowchart, (FIG. 15) agent movement logic is presented with the help of different pedestrian library blocks. This flowchart starts with agent generation based on class schedule, agent movement in the continuous space considering optimal physical distancing policy and finally agent removal from the simulation at the end. Statistics from two perspectives namely, risk and logistics, were reported from the simulation flowchart (starting from pedGoToClass5 to pedGoToSinkRest1).

Different policy implementations were tested in different classroom settings (FIG. 16) to find out the best seating arrangement with lower perceived risk. Some of the tested policies are zonal exit, non-zonal exit, 50% maximum occupancy, 50-person maximum occupancy.

To prevent the uncoordinated movement of pedestrians, a realistic social force model including individual physical and psychological characteristics and the collective herd instinct is employed in this study. It involves individual physiological, psychological characteristics and collective herding instinct. The main components of the social force are comprised of:
Pedestrian's self-consciousness
Force from other pedestrians
Force from environment (walls, doors, objects)

Evaluation of best routing and seating configurations: Different scenarios for entry-exit policies have been performed under the utilization of multiple route choice models. By varying initial conditions, we evaluated different route choice and resource allocation (seat) models to find out the best policy recommendations for safe and efficient use of the facility.

Shortest Path (Dijkstra's Algorithm)

Dijkstra's algorithm is an algorithm for finding the shortest paths between two nodes. When a student agent enters the classroom, seats from the available resources are assigned based on Dijkstra's shortest path algorithm considering the entry point of the student. Similarly, when the class ends, student's exit door is selected based on the shortest distance from his current location.

Physical Distancing

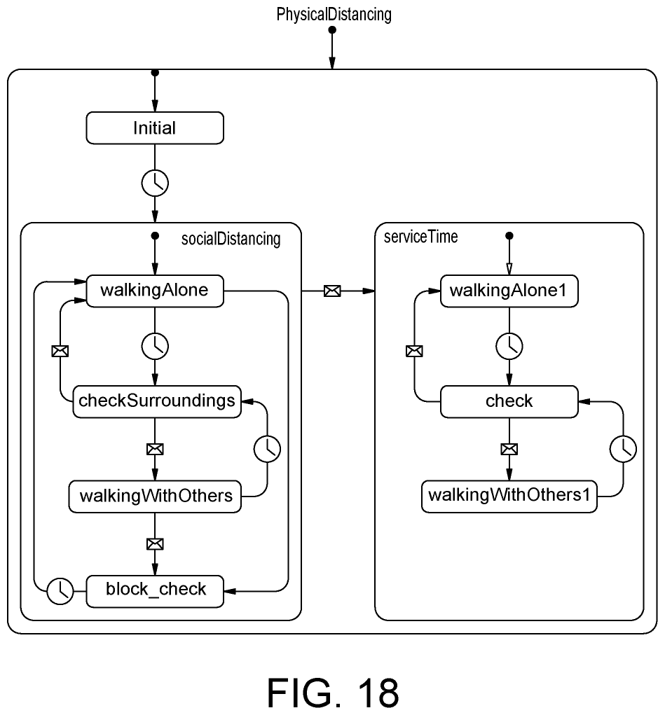

Deadlock—A deadlock detection algorithm is configured to dynamically detect & resolve deadlock situations (e.g. when pedestrians passing through a narrow lane and become immobilized due to other pedestrian movement from the opposite direction) while practicing social distancing. It will turn off social distancing for certain seconds (depending on aisle width, deadlock duration) to ensure smooth realistic pedestrian movement (see FIG. 18).

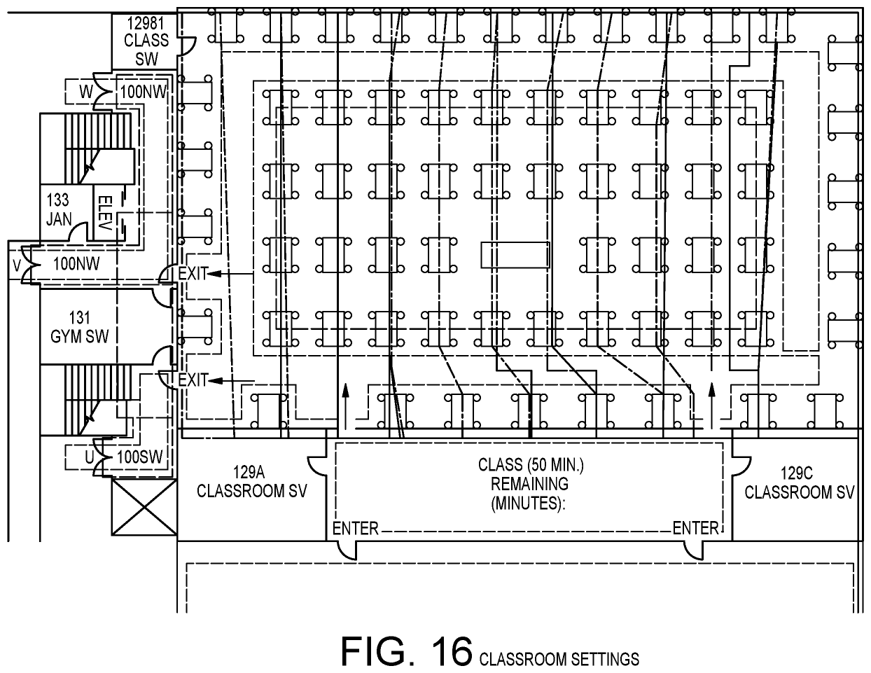

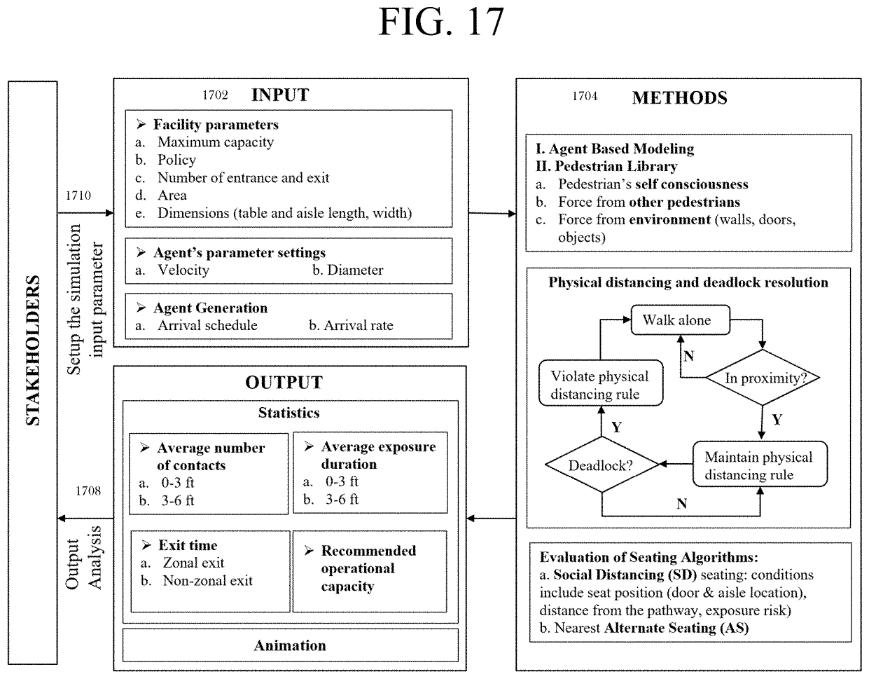

Output: The Output of the model is shown in FIG. 17 and comprises the below.

A realistic animation of the movement of agents and statistical results related to agent's interactions are displayed on the dashboard. Statistics from two perspectives namely, risk and logistics performance were reported from the simulation.

Performance

Exit Time—
For safe operations of in-person class, exit time was reported based on all agents exit time from the class environment.
Different exit path restriction policies were tested to find out the best policy recommendation for the classroom facility. (e.g. single door entry/exit, multiple door entry/exit)
Zonal and non-zonal (FIG. 12) exit policies were tested for a safer and quicker exit strategy

Simulation runtime performance (model execution time, number of iterations and memory allocation) will be used to show the difference of model performance under different model configurations.

Risk

Since agents can spread the virus before they know they are sick, it is of utmost importance to stay at least 3 feet (6 feet) away from others when possible. That is why, two important matrices of the simulation are average contact time and average contact number within 0-3 feet & 3-6 feet range. Exit time and risk parameters for a simulation of a collaborative classroom setting is shown in FIG. 19.

In both average contact time & contact number calculation, agents risk parameters were continuously calculated based on the number of contact & exposure duration within 0-3 feet & 3-6 feet range. In every time interval (5 sec), pedestrian agent search for other pedestrians within his 0-3 feet and 3-6 feet diameter range. Whenever, someone enters within this range, the model stores the first interaction time into that time bucket (0-3 feet, 3-6 feet). As soon as the intruder pedestrian leaves this physical distancing range, the model stores the final time & calculate total exposure duration caused by that specific individual. During the whole simulation time, if one individual is exposed by multiple pedestrians at the same time with different exposure duration, the algorithm can detect and collect pedestrian specific exposure duration to mimic a real-world scenario. Average contact time & average contact number for an individual agent are shown in FIG. 15.
Average Contact Time
   0-3 feet
   3-6 feet
Average Contact Number
   0-3 feet
   3-6 feet

Simulation Modeling—Purpose

The purpose of the model is to mimic and evaluate different policies (viz. entry and exit policy, seating policy, and seat layout) involving indoor activities, and devise the most appropriate policies, which can minimize the contact-caused risk to the organization in the event of a pandemic. In this analysis, classrooms have been considered for the case study to ensure the extensibility of the analysis for other indoor venues (e.g., cinemas, auditoriums, indoor sports fields, seminars, and airlines). Evaluation of policies using the model is primarily based on statistical results of risk and logistic parameters. We utilize total time for all students to leave the classroom (exit time) as the logistical parameter in the model. As the perception of risk varies with the situation, based on COVID-19 pandemic context, the average amount of time an agent spends within proximity (within 0-3 feet and 3-6 feet range) of the other agents (i.e., average exposure duration) and the average number of contacts an agent has been with other agents is considered for risk evaluation. We also introduced a physical distancing and deadlock resolution framework into the embedded social force model to ensure realistic pedestrian behavior during the simulation. In addition, an automatic Social Distancing (SD) seat selection algorithm was implemented and tested against the nearest Alternate Seating (AS) seating algorithm in the simulation model.

Simulation Modeling—Process Overview

As shown in FIG. 17, at the model startup, the initialization parameters 1702 (e.g., facility parameters, agent parameters, and agent generation data) are assigned based on modeling requirements from the user. In a pedestrian dynamics' context, the Anylogic pedestrian library, was used to generate student agents that follow the social force algorithm. In the next step 1704, student agents are assigned seats in the classroom based on maximum distance from the doors and pathways. A comparative analysis 1706 for the performance of different seat selection methods has been performed to evaluate entrance and exit policies along with their associated contact and exposure duration. During the simulation, physical distancing and deadlock resolution mechanisms have been deployed to ensure a safe and realistic model performance. At the end of the simulation 1708, a dashboard has been designed to provide users with an overview of the output statistics. Finally, the user is given the option to change the input configuration 1710 depending on the output statistics to identify the optimal policy and classroom setup.

Simulation Modeling—Agents (Parameters and Variables)

The primary focus of the analysis is to mimic the movement of the pedestrians within indoor spaces realistically. Therefore, ‘student agents’ have been used to perform the movements. Table 6 lists the parameters and variables of the student agent. To define the configuration and setting of the simulation environment, various parameters and variables are considered based on requirements provided by the organization, such as classroom capacity, student agent arrival rate, duration of the class, duration of the break, total entrance time, and total exit time. All the model relevant information (e.g., class schedule, class time, classroom capacity, classroom dimension, entrance and exit policy) used in the analysis was provided by concerned university authorities to represent the classroom environment.

TABLE 6
Agents, Parameters, and Variables

| Agent | Parameters | Variables |
|---|---|---|
| Student | Initial seat | Agent diameter, deadlock duration, |
| | location, agent | color, start of entrance time, end of |
| | diameter, | entrance time, start of exit time, end |
| | velocity | of exit time |

Simulation Modeling—Social Force Model and Pedestrian Library

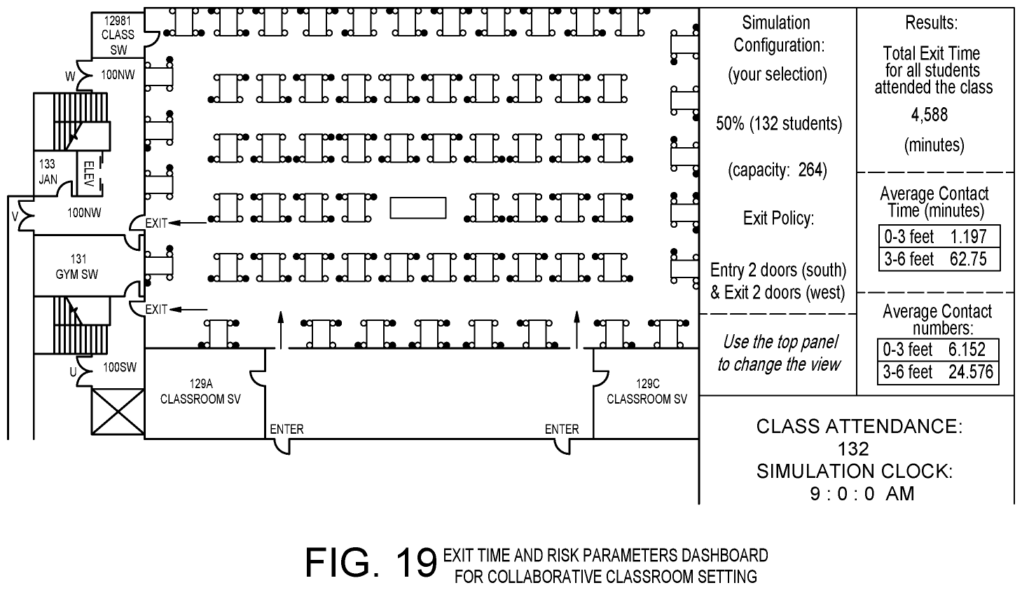

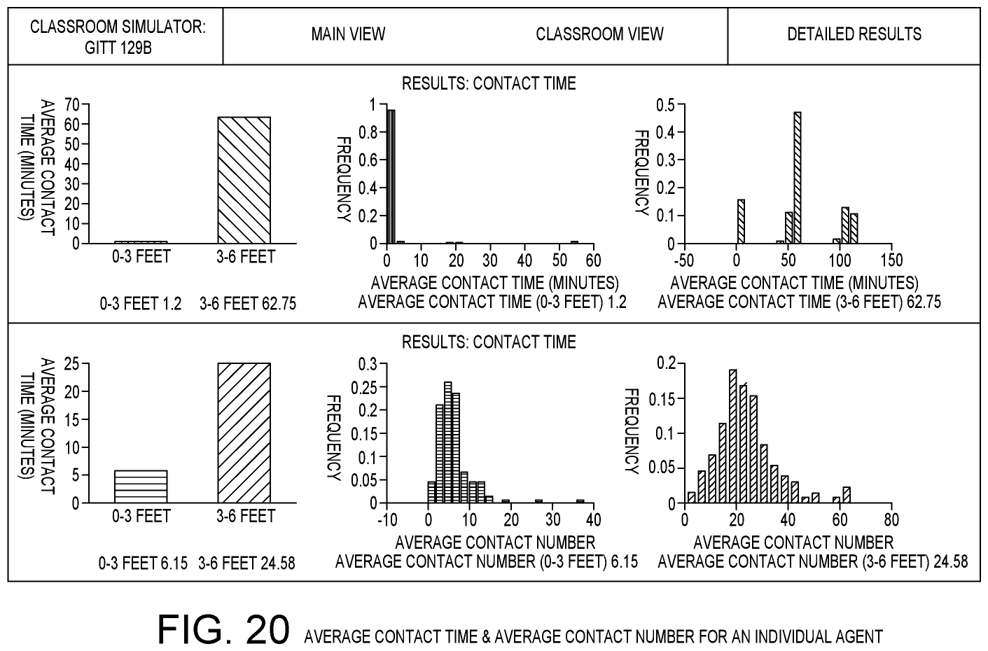

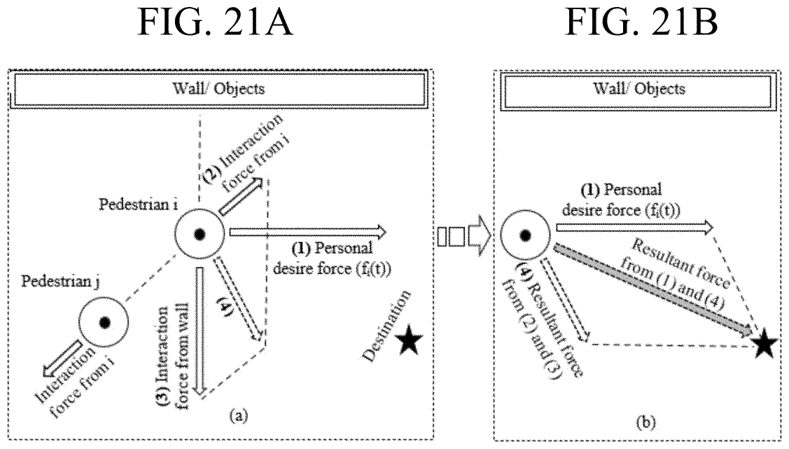

Student agent behavior was modified within the Anylogic pedestrian library by modeling physical distancing and deadlock detection and resolution mechanisms, and was used to assess capacity and throughput, identify bottlenecks caused by pedestrians, and execute planning within a public area. The movement of the pedestrians within the environment is governed according to the social force model. Each agent within the simulation utilizes the shortest path to perform the movement and avoid collisions with other objects (e.g., walls, desks, and other pedestrians). The pedestrian behavior is defined using a block diagram, which specifies the movement patterns and destinations across space. The physical environment is comprised of the space markup elements such as walls, service points, and attractors. To prevent the uncoordinated movement of pedestrians, a realistic social force model including individual physical and psychological characteristics and the collective herd instinct is employed in this analysis. The main components of the social force are (a) pedestrian's self-consciousness, (b) force from other pedestrians and (c) Force from the environment (walls, doors, objects) (FIGS. 21A and 21B).

The governing equation for the social force model implemented in the pedestrian library is as below:

$${m}_{i} ( \frac{{dv}_{i}}{dt} ) = {m}_{i} ( \frac{{v}_{i}^{0} ( t ) {e}_{i}^{( 0 )} ( t ) - {v}_{i} ( t )}{{τ}_{i}} ) + {\sum }_{j ( \neq i )} {f}_{ij} + {\sum }_{w} {f}_{iw} \qquad ( 12 )$$

Pedestrian's behavior is depicted by desired speed (v⁰ᵢ(t)), direction (e⁰ᵢ(t)), and interactions with other pedestrians (fᵢⱼ), walls and objects (f_{iw}). To better understand the forces acting on the pedestrian j, we developed two diagrams that illustrate the forces and their resulting vectors. The first term on the right side of equation 13 represents the pedestrian's self-consciousness (force component (1) in FIG. 21A), while the other two terms represent the interaction force from other pedestrians (force component (2) in FIG. 21A, walls, desks, and objects (force component (3) in FIG. 21A). Force component (4) in FIG. 21A is the resulting force of (1) and (2). The green dotted arrow in FIG. 21B represents the resultant force (1) and (4), which guides the agent to its destination.

Simulation Modeling—Physical Distancing

Physical distancing, also called ‘social distancing’, means keeping a safe space between two individuals belonging to a different cohort. It has been one of the essential factors and practiced policies during the COVID-19 outbreak due to its effectiveness in reducing disease transmission among humans. In a regular setting, efficient conduct of daily activities often requires in-person interaction among individuals of different ages, races, races, and genders. That is why it is essential to maintain physical distancing with other daily preventive measures in all environments and activities involving people.

The concept of physical distancing has been a focus area in recent works due to the high volatility, high contact-based spreading, and mortality rate of the COVID-19 virus. Correspondingly, in simulation modeling, physical distance is a new field and can provide a significant contribution to the original Social Force Model.

In this analysis, we introduced a new physical distancing framework to ensure a safe boundary between student agents. While most public health officials recommend 6 feet distance between people, a review of 172 studies from 16 countries concluded that 3 feet distance is effective with proper face masks and other safety measures. Another study on 251 school districts of Massachusetts public school districts, encompassing 540,000 students and 100,000 K-12 staffs, who attended a 16 week in-person learning program did not show significant difference in the number of Covid-19 cases under three feet of social distancing, as opposed to six feet measures. In light of these studies, the nation's top infectious disease experts are planning to validate three feet of social distancing as the safety measure for reopening schools in coming days. So, in this analysis, we considered 3 feet mandatory physical distancing policy considering proper face masks and a sanitized classroom environment.

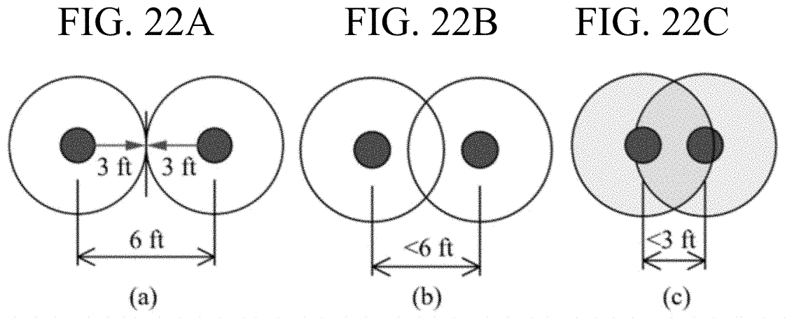

In this analysis, the physical distance has been implemented by dynamic changes in the agent's diameter during interaction with other agents illustrated in FIGS. 22A-22C. Each student agent ensures 3 feet physical distance from others in the class environment (FIGS. 22A-22B). Whenever someone comes within 0-3 feet of a student agent, that student's distance measure is violated, and the physical distancing circle turns red (FIG. 22C). As the modeled environment represents a classroom where the majority of the students are in the mid-20s, a 0.65 feet radius cylinder (i.e., 1.3 feet diameter) has been considered for each student agent to represent the average human shoulder width. In addition, for the visual representation of an agent, a dummy cylinder of the size of the student agent was considered.

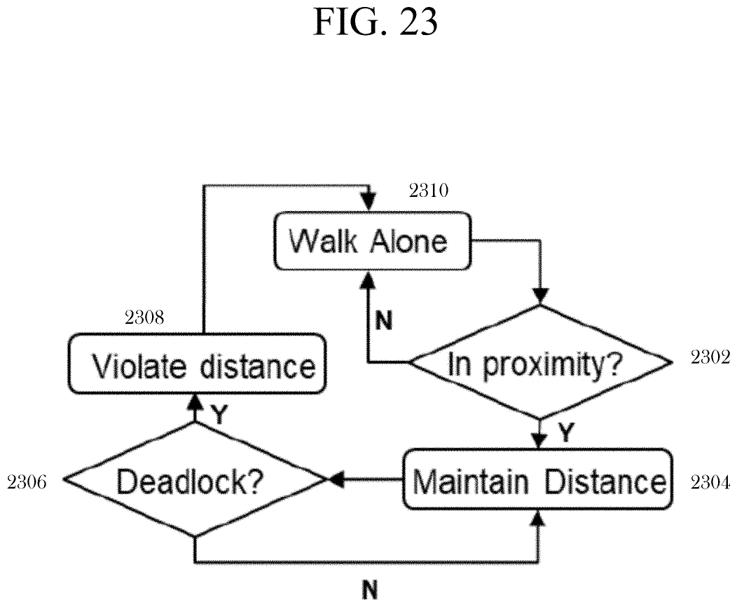

When moving in a virtual environment, the student agent constantly searches its surroundings (FIG. 23) to detect at “In Proximity?” 2302 any other student agents within a radius of 3 feet (6 feet in diameter), thus violating the physical distancing constraint. As a result, at “Maintain Distance” 2304, the diameters of the agents increase to 3 feet from 1.3 feet whenever another student is inside that 3-feet radius (6-foot diameter) zone. The system also checks for deadlock situations at “Deadlock?” 2306 to determine whether there is a distance violation 2308 that would necessitate walking alone 2310. However, visualizing a change in the size of a human body due to interaction is implausible. To avoid this, we used the constant dummy cylindrical circle as a visual representation while changing the actual agent diameter and hiding it from the simulation screen. Due to the default social force, this diameter change in agent size results in a body compression and sliding friction force by impeding the tangential motion of the agents. As a result of this force, both agents move away from each other and maintain 3 feet physical distance in the simulation. Eventually, by changing the agent's actual diameter in the simulation, we use the inherent social force to exert a repulsive force that pushes the agents apart but keeps the visual representation (dummy diameter) of the agent the same as before.

The problem occurs in a classroom-like environment, where many obstacles, such as walls, desks, and chairs are present. Analogous to the interaction force between pedestrians, physical objects also trigger interaction forces on student agents when the agent body is close to the obstacles. This leads to sudden changes in agent diameter due to physical distance violation, thus causing instability in the agents' movement due to dynamically changing distance from other agents and objects. For example, with a repulsive force applied to push the student agent backwards, the student agent may also get a forward push if there are other obstacles near the backward direction. That eventually leads to repeated push and vibrations. Suppose the student agent moves in a congested environment surrounded by walls and objects. In that case, this instability will be exacerbated by repeated changes in diameter, which is common in classroom-like environments.

Since the pedestrian library of AnyLogic 8.5.1 was not developed for physical distancing modeling, because of the sensitivity of the social force algorithm used in the library, the model does not perform well when the agent diameter increases and changes to 3 feet (which is unreasonable for a human diameter). To overcome these challenges, we incorporated the following approaches:

Different Scaling

In AnyLogic, the scaling ratio is set as the ratio of the animated pixels to the physical unit of length. In the simulation, we specify the unit of length to pixel correspondence to represent the object of the real-world. Usually, the scale is set to a fixed unit at both the animation level and agent level. To overcome the unrealistic crowd behavior caused by the diameter change, we used different scales in the main simulation interface (animation scale) and the student agent (actual pedestrian scale) in the model. However, we increased the student agents' diameter by the factor of scale ratio to maintain consistency in both simulation main and student agent. By implementing this scaled-down method in the student agent, we generated realistic crowd movement behavior throughout the simulation.

Time Step

By setting the time step parameter to lower values, simulation can track student agents' movement more precisely. We used a time step of 0.05 seconds in the simulation, which enabled a smooth student agent movement but made the model computationally expensive.

Simulation Modeling—Deadlock

It is imperative to represent realistic movement of students within the classroom setting. The dynamic changes in the diameter of the pedestrian cylinder in a social force model often led to blocking or deadlocking the pedestrian movements in narrow pathways if a resolution mechanism is not provided explicitly. Hence, in order to represent realistic human intervention nature, it is important to devise a resolution technique to handle deadlock situations. Deadlock is one of the commonly used situations in distributed simulation, automated manufacturing system, and communication networks. A deadlock occurs when a group of processes intend to acquire the same resource, but the resource requests cannot be satisfied due to the limited resource. In the analysis, initially we have observed some deadlock situations within the narrow pathways of the classroom. So, we devised a deadlock detection and resolution methodology to represent more realistic student movements (e.g., human interventions).

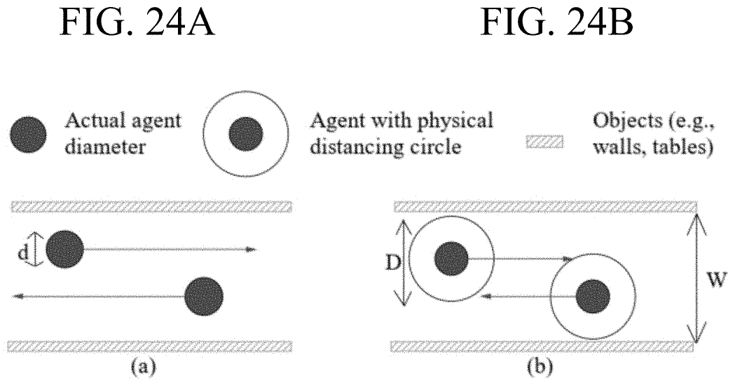

A deadlock could occur in the simulation model due to a scarcity of the resource pathway space. Due to the abundance of desks and chairs, and physical spacing requirements in the classroom, the classroom space is packed, and students' movements are more restricted than the non-pandemic situation. When student agents moving in opposite directions intend to pass each other on a narrow path while adhering to the physical distancing requirement, a deadlock situation may arise. Suppose the pathway width (W) is less than the sum of both agents' physical distancing circle diameters (D). In that case, the student agents will not pass each other due to physical distancing restrictions, resulting in a deadlock (FIG. 24B). However, if there were no physical distancing restriction in the model, students' agents would be able to move freely without experiencing any deadlock since the pathway width (W) is more than the total of actual agent diameter (d) (FIG. 24A). As such, deadlock situations would rarely occur for non-pandemic scenarios, without requiring physical distancing. For pandemic scenarios, the deadlock may occur, and the detection and resolution algorithm is applied to represent the real human intervention behavior.

In some situations, a wide pathway can also create a deadlock situation due to multiple student agents' presence at the same time. Therefore, it is important to understand and devise a deadlock resolution technique to model realistic pedestrian behavior. In the simulation model, most of the space is occupied by the physical distancing circle of each agent. One feasible solution to avoid deadlock situation is by disabling the physical distancing algorithm for a particular instance to allow both student agents to share the available pathway space. The first step of this deadlock resolution is to identify a deadlock situation. A deadlock situation cannot be declared when an agent is not moving for a certain duration. Hence, simply labeling zero velocity as a deadlock situation will misclassify a student seating in the chair as deadlock. In this model, deadlock logic was formulated by considering several information during the simulation runtime including the time of the incident (viz. class time or break time), the number of student agents involved in the situation, the width of the pathway, and the amount of time agents spent in the stagnant situation. The combination of all the information provided insights to accurately classify the deadlock instance and facilitate the participating agents to turn off the physical distancing mechanism for that specific moment (i.e., human intervention nature in a real-world setting).

Simulation Modeling—Evaluation of Seat Choice Algorithms

Another important aspect investigated in this analysis is to find an optimal seating policy by minimizing the possible number of contacts and exposure in a classroom environment. Thus, we have tested two different seating methods in the simulation under different policies and classroom setting to find the best seating arrangement. The performance of seating policies was evaluated based on three types of simulation output: exit time, average exposure duration, and average number of contacts.

Different room configurations have been considered for testing seat selection policies. In order to represent the general functional room of educational institutions, GITT129B from the Ina A. Gittings Building at the University of Arizona is used as a case study. We considered this regular classroom and tested different layouts (e.g., collaborative, traditional) and exit policies (zonal exit, non-zonal exit) for different occupancy level.

The data pertaining to the dimensions of the classroom, desks, chairs, width of the pathways, the distance between seats, location, and the number of entrances and exits, teacher's corner, and available technologies were considered to design the classroom within the simulation. Once the appropriate layout and design have been implemented, the seats for seat selection evaluation must be labeled prior to testing different policies. Hence, the appropriate notations have been formulated to devise a unique label for each seat within the classroom.

The notations for seat labeling are shown below:
r, r′=row number of the seat, ∀r,r′ϵR
p,p′=upper or the lower section of the corresponding row,
p,p′=1 represents upper section and, p,p′=2 represents the lower section
c,c′=column number of the seat, ∀c,c′ϵC
q,q′=right or left side of the corresponding column q,q′=1 represents right side and q,q′=2 represents left side.
R=Set of rows, {1,2,3, based on classroom layout}
C=Set of columns,{1,2,3, based on classroom layout}

The data pertaining to the dimensions of the classroom, desks, chairs, width of the pathways, the distance between seats, location, and the number of entrances and exits, teacher's corner, and available technologies were considered to design the classroom within the simulation. Once the appropriate layout and design have been implemented, the seats for seat selection evaluation must be labeled prior to testing different policies. Hence, the appropriate notations have been formulated to devise a unique label for each seat within the classroom.

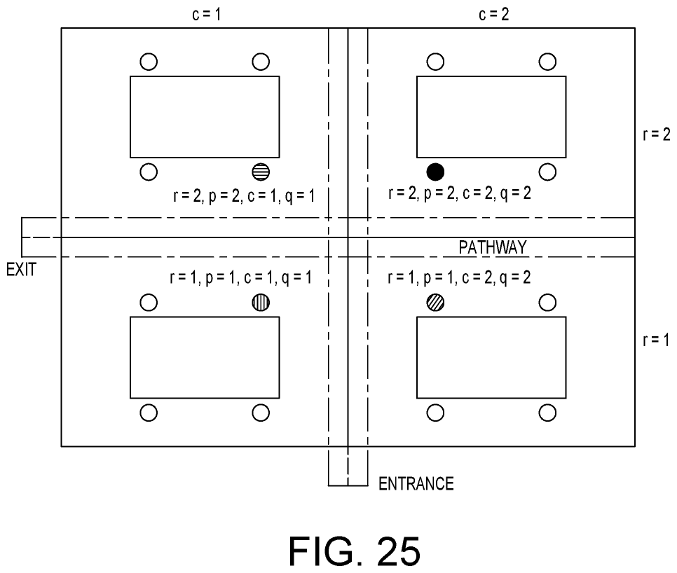

FIG. 25 shows the seat labeling procedure through visual illustration. Spatially, the black colored seat has the row value (r=2) and column value (c=2), alternatively black colored seat is located at the intersection of the 2nd row and the 2nd column. The next step is to locate the seat (e.g., right, or left side of the column and upper or lower section of the corresponding row) based on positional value from the corresponding column and row. For example, the black seat is on the lower section of the row (p=2) and on the left side of the column (q=2). So, that black seat can be labeled as (r=2,p=2,c=2,q=2). Similarly, the green seat can be labeled as (r=1,p=1,c=2,q=2).

The distance values have been derived by using Euclidean distance formula between two seats as shown in Equation 13:

$$Euclidean distance , D = \sqrt{( {X}_{r , p , c , q} - {X}_{{r}^{′} , {p}^{′} , {c}^{′} , {q}^{′}}^{′} ) + ( {Y}_{r , p , c , q} - {Y}_{{r}^{′} , {p}^{′} , {c}^{′} , {q}^{′}}^{′} )} \qquad ( 13 )$$

Based on the unique labels generated for each seat within the classroom, two seat selection policies have been tested in this analysis, (a) proposed SD seating, and (b) AS seating.

Seat Selection Method 1: SD Seating

This method proposes a seating policy to make appropriate seat assignments to students to ensure the appropriate social distancing while attending the class. The policy works by associating penalty value to each seat based on the distance from entrance and exit doors and distance from the nearest pathway.

Seats located near the doors are subject to higher penalties because these seats are close to the entrance or exit door and have a higher potential for contact and exposure. Conversely, seats from the farthest corner of the classroom are least penalized due to the lower contact and exposure risk because of their location.

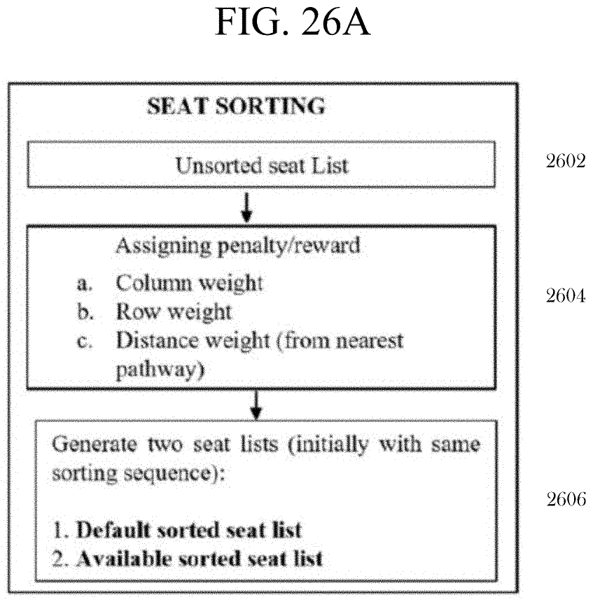

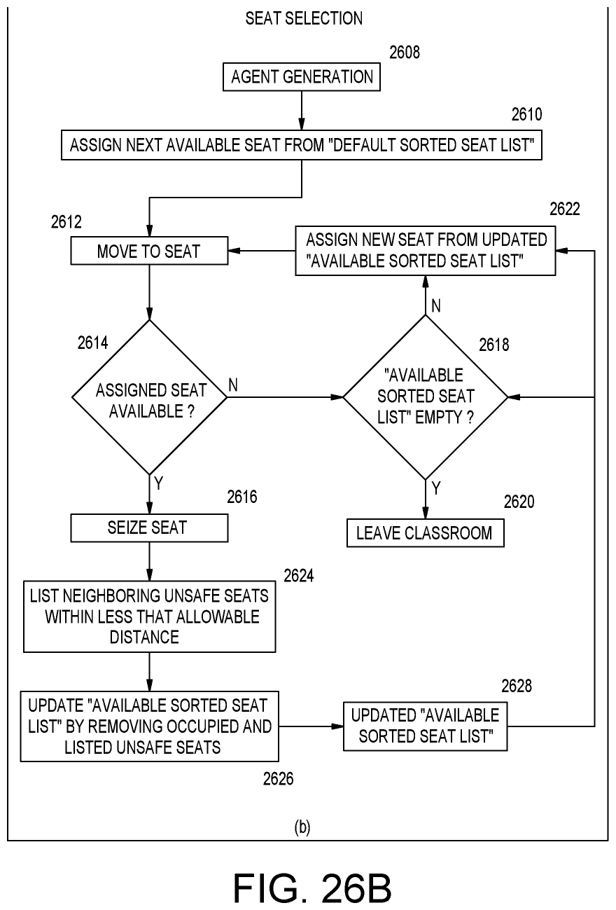

The proposed SD seating policy consists of two segments: seat sorting and seat selection. As shown in FIG. 26A, the unsorted seat list 2602 is received by the software. All seats are sorted 2604 based on the column, row, and distance weights on model startup. Initially, the same sorted seat list was used to create two identical seat lists 2606 (default sorted seat list, available sorted seat list). Sorting is based on a seat's desirability: the seat with the lowest penalty is at the top and the seat with the highest penalty at the bottom). Seats were assigned to the students from the default sorted seat list upon creation based on the first come first serve basis.

As shown in FIG. 26B, following agent generation 2608, as the student agent's seat is assigned 2610, the student goes to the designated seat 2612 by following 3 feet physical distancing guideline. On arrival to the seat location, each agent confirms whether the assigned seat from the ‘default sorted seat list’ is also available in ‘available sorted seat list’ 2614. If the seat is available in both lists 2614, the student agent occupies the allocated seat 2616 and the ‘available sorted seat list’ is updated 2626 by removing the occupied seat. Furthermore, upon arrival at the assigned seat, the algorithm immediately searches for the other available seats in the area that violates the 3 feet distancing rule. Once those distance violating seats are listed 2624, all of them are subsequently removed from the ‘available sorted seat list’ 2626. Eventually, all the seats will be either assigned or removed by the algorithm due to the physical distancing measures and once the ‘available sorted seat list’ is empty 2618, the new students will be advised not to enter the classroom 2620 due to the unavailability of safe seats.

Seat Penalization

The rows or columns with a smaller Euclidean distance from the doors possess a higher penalty for potentially high exposure risk and physical distancing violation as those seats have high pedestrian contact potential. To address this effect, we introduce βr, γc, δ(r,c) as weights to address the importance of minimizing the number of students on seats closer to the door area.

Here, βr is the penalty imposed on a desk in row r due to the presence of an entrance or exit door in parallel to that row. γc is the penalty imposed on a desk in column c due to the presence of an entrance or exit door in parallel to that column. δ(r,c) is the average penalty imposed on a desk due to the presence of an entrance or exit door in parallel to that row and/or column.

Consider a classroom with a single door that serves as both an entrance and an exit for all the students. The door is located near and in parallel to the first row (r=1). So, the distance between the door and the rows increases with the increase of row numbers (e.g., r=2,3,4,5). Intuitively, students seated on the first row (r=1) will have higher probability of getting in contact with entering students than the students seated in the rows of higher value (e.g., r=2,3,4,5). So, βr should be inversely proportional to the row number or βr∴1r. Similarly, considering an entrance/exit door near and in parallel to the first column (c=1), γc should be inversely proportional to the column number or γc∴1c. Now, if there are two doors in a classroom, one near and parallel to the first row (r=1) and the other close and parallel to the first column (c=1), the overall penalty of a desk position should be proportionate to the average of the associated row and column penalties.

$${δ}_{( r , c )} ∝ \frac{{β}_{r} + {γ}_{c}}{2} \qquad ( 14 )$$

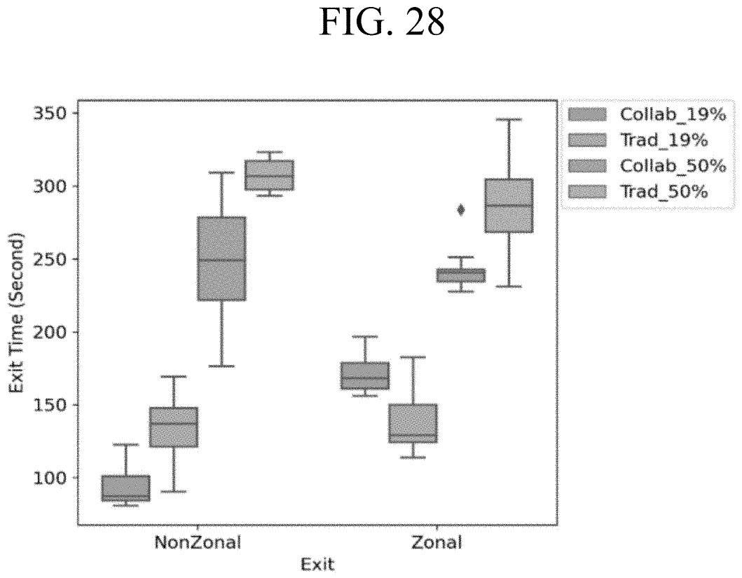

Scenario 1: Some doors are utilized more frequently than others in certain scenarios, prompting the application of a higher penalty weight to some doors. If we consider a door (FIG. 28A) near and parallel to the first row (door 1), which is more frequently used for entrance and exit operations than the door in parallel to the first column (door 2), which is only used for exit operations, intuitively we should assign a higher penalty to the seats located near door 1 than door 2. To address this issue, we took the following approach:

$$\begin{matrix}{β}_{r} = {( \frac{1}{r} )}^{\frac{1}{{θ}_{1}}} , ∀ r ∈ R , {θ}_{1} ∈ {Z}^{+} & ( 15 ) \\ {γ}_{c} = {( \frac{1}{c} )}^{\frac{1}{{θ}_{2}}} , ∀ c ∈ C , {θ}_{2} ∈ {Z}^{+} & ( 16 )\end{matrix}$$

We can use this method to assign different penalty weight factors based on a desk's row and column positions. For instance, in this case (FIG. 28A), the location of desks 2 and 4 can be considered. At first look, it may appear that both desks should have the same penalty factor because they are adjacent to the door. Desk 2 should, however, have a greater penalty and so be less desirable than desk 4 because door 1 is utilized more frequently than door 2. That requirement can be easily addressed by considering different values for θ1 and θ2. Direction towards which the room has higher traffic flow should have higher θ value compared to direction towards which the room has lower traffic flow. Since door 1 has higher traffic flow in column wise direction (between column 1 and 2), we consider θ2=10 to calculate column wise penalty γc and consider θ1=5 to calculate row wise penalty factor βr.

For desk 2, r=1, c=2, so we get: β1=(1/1)^{1/5},=1, γ2=(12)110,=0.933. So, from equation 3, δ1,2=1+0.9332=0.966.

For desk 4, r=2, c=1, so we get: β2=(12)15=0.871, γ1=(11)110=1. So, from equation 3, δ2,1=0.871+12=0.935.

So, for desk 2 we are getting penalty factor 0.966 whereas for desk 4 penalty factor is 0.935. So, desk 2 is more penalized and thus less desirable than desk 4. Similarly, we can get the penalty factors for other desks from Table 7 as below. From Table 7, we get an order of desk's desirability shown as D (desk number):PF (penalty factors from low to high): D(12): PF(0.826), D(11): PF(0.845), D(9): PF(0.849), D(8): PF(0.868), D(10): PF(0.879), D(6): PF(0.883), D(7): PF(0.901), D(5): PF(0.902), D(3): PF(0.948), D(4): PF(0.936), D(2): PF(0.966), D(1): PF(1).

TABLE 7
Seat Penalization Score (βr, γc)

| Row (r)/Column | | |
|---|---|---|
| Number (c) | θ = 5 | θ = 10 |
| 1 | 1 | 1 |
| 2 | 0.870550563 | 0.933032992 |
| 3 | 0.802741562 | 0.89595846 |
| 4 | 0.757858283 | 0.870550563 |

Scenario 2: Consider another scenario (FIG. 7. (b)), in which a room has two doors, one of which is near and parallel to the first row (r=1) and the other of which is near and parallel to the last row (r=4). In that case, the row-wise penalty for rows 1 and 2 should be the same as row 4 and row 3. The penalty equation for each desk's row position will thus be:

$$\begin{matrix}{β}_{r} = {( \frac{1}{r} )}^{\frac{1}{{θ}_{1}}} , ∀ r ∈ R , \backslash \{ r \geq {r}^{′} / 2 \} , {θ}_{1} ∈ {Z}^{+} & ( 17 ) \\ {β}_{r} = {( \frac{1}{( {r}^{′} + 1 ) - r} )}^{\frac{1}{{θ}_{2}}} , ∀ r ∈ R , \backslash \{ r \geq {r}^{′} / 2 \} , {θ}_{2} ∈ {Z}^{+} & ( 18 )\end{matrix}$$

Here, r′=maximum row value (for this example, r′=4). Similarly, if the doors were parallel to the first and last columns, we may write a similar equation for column wise penalty γc and determine the weighted penalty δ(r,c) from Equation 14.

$$\begin{matrix}{γ}_{c} = {( \frac{1}{c} )}^{\frac{1}{{θ}_{3}}} , ∀ r ∈ C , \backslash \{ c \geq {c}^{′} / 2 \} , {θ}_{3} ∈ {Z}^{+} & ( 19 ) \\ {γ}_{c} = {( \frac{1}{( {c}^{′} + 1 ) - c} )}^{\frac{1}{{θ}_{4}}} , ∀ c ∈ C , \backslash \{ c \geq {c}^{′} / 2 \} , {θ}_{4} ∈ {Z}^{+} & ( 20 )\end{matrix}$$

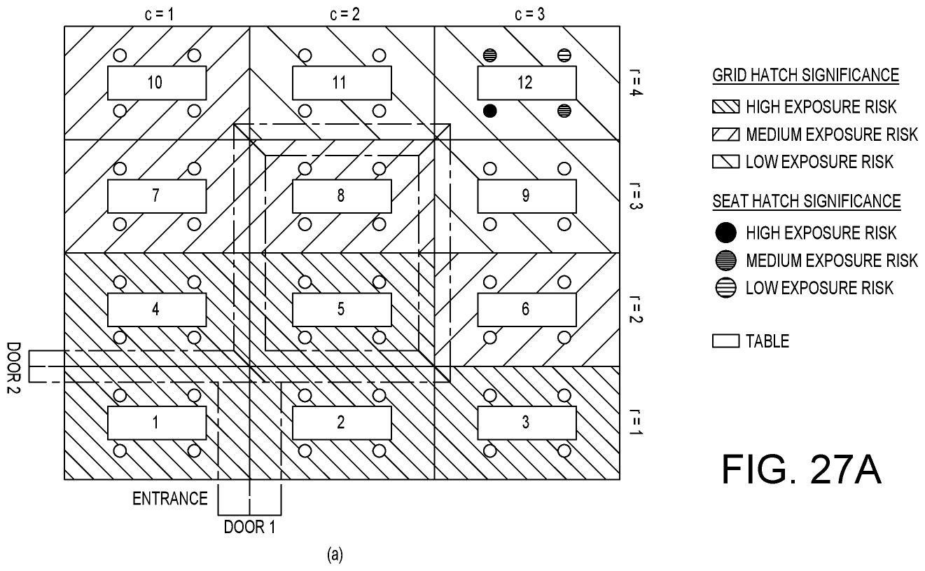

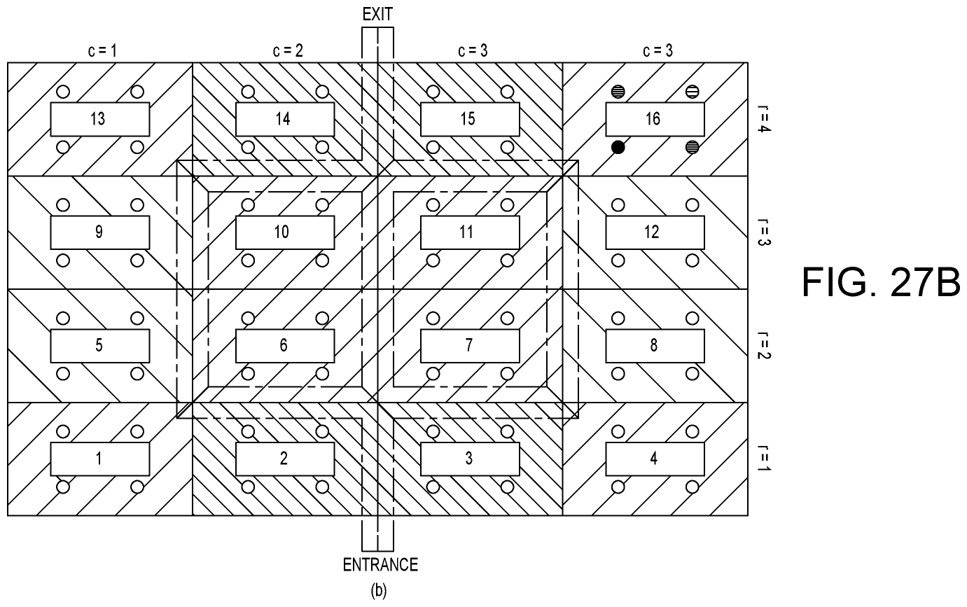

In the demo classroom settings, a graphical representation of seat preference with the help of color grading has been shown in FIGS. 27A and 27B. In the room (FIG. 27A), we have an entrance on the lower-left corner and an exit on the left. The aisle (pathway) surrounds the classroom. The seats nearest to the door area are more prone to exposure due to the frequent movement of the student agent in that area. Hence, the model divides the room into blocks based on rows and columns. As shown in FIG. 27A, the door-side blocks are more reddish, and when we move from the lower-left corner to the right or up, its color will gradually disappear. It can be easily understood that the lower-left corner block is at a higher exposure risk, while the upper right corner block is at a relatively lower risk.

The penalty for a specific row and column intersecting desk is described in the seat penalty section above. Now, considering a particular desk (e.g., upper right corner), there are multiple seats, and the model ranks them according to the distance from the closest path. Understandably, the seat closest to the aisle (red) should have the most exposure and thus rank lowest, while the seat with the greatest distance should have the least exposure (green) and thus rank highest. So, the model iterates through all desks, ranks them based on row and column penalty factors, and then ranks individual seats within a desk based on pathway distance. Then, when a student agent enters the classroom, the student will be allocated a seat from the ranked seat's list in order of highest to lowest based on availability.

Seat Selection Method 2: AS Seating

The AS seating policy was tested in the simulation model to compare the risk associated with this policy against the proposed SD seating. In this policy, every alternate seat is assigned to the incoming student based on the shortest distance from the entrance door. The classroom simulation in this analysis has two entrance doors on the front side of the room and a teacher's desk in the front half of the room. A study shows that the student learns better when they seat in proximity to the teacher compared to the distant seat position. So, it is imperative to analyze the nearest seat policy in this analysis. It ensures proximity to the teacher facilitating a good learning environment and represents a real-world classroom situation. To minimize contact among students, we removed every alternate seat in this seating policy. In the AS policy, the simulation uses Anylogic's default Dijkstra's algorithm to find the shortest path from the entrance to a seat. The shortest route between two nodes is calculated using Dijkstra's algorithm. The first node represents the student's entrance door, while the second node denotes the student's seat location. When a new student agent arrives at the entrance door, this algorithm sorts all the available seats based on the shortest distance from that specific door and assigns the nearest seat to the student. Once the student sits on that designated seat, the AS algorithm removes the next seat to ensure a safe classroom environment. Eventually, this algorithm finds the nearest seat from the available seat list and assigns it to the new student when the next student arrives.

Simulation Configuration

Different simulation configurations facilitate the verification and validation of a simulation model. Hence, in the analysis, the most important aspect of the validation process for different indoor settings is to test the model under different simulation configurations. The tested simulation configurations are described in Table 8.

TABLE 8
Simulation Configuration

| Properties | Configurations |
|---|---|
| Layout | a. Traditional (FIG. 8 (a)) |
| | b. Collaborative (FIG. 8 (b)) |
| Facility type | a. Regular classroom (FIG. 8 (a), (b)) |
| | b. Meeting room (FIG. 8 (f)) |
| | c. Auditorium (FIG. 8 (e)) |
| | d. Athletics (dance class. FIG. 8 (d)) |
| Exit rule | a. Zonal (FIG. 8 (b)) |
| | b. Non-zonal (FIG. 8 (c)) |
| Maximum allowable | a. 50% (132 students) |
| capacity | b. 19% (50 students) |
| Seat choice policy | a. SD policy |
| | b. AS policy |

Different types of coursework require different nature of classroom parameters such as classroom type and maximum allowable occupancy of the class. For instance, in a collaborative classroom, students can sit around the workstation, facilitate group discussions, collaboration, and actively participate in the learning process. Conversely, traditional classrooms allow the student to have a good view of the front of the room and enables the instructor to control the students. Correspondingly, depending on the need, a room can be used as a classroom, meeting room, office room, auditorium, or athletic program (e.g., dance class). All these rooms have different use cases, different numbers of entrances and exits, and different numbers of seats depending on the room's functionality. Hence, the model was tested on all these various configurations of facilities to ensure validity, robustness, and assistance to the university policymakers.

During the exiting movement of the crowd, close contact or interaction between student agents will increase the risk of viral infection. Globally, there is increased awareness of imposing different door entry and exit rules to reduce the cross-contact among people. Across the country, many large supermarkets, restaurants, and office buildings have adopted this rule by restricting entry and exit policies. Correspondingly, for the flow of large crowds in and out of theatres, classrooms, meeting rooms, prayer rooms, and auditorium-type places, it can be a good strategy to divide the entire space into different zones and utilize zonal policies for entering and exiting into the indoor spaces. Individuals from a zone will move towards the next zone or exit only if the next zone is empty. By doing this, we can avoid congestion near exit areas and reduce contact among people.

In the context of the pandemic, there are increasing concerns about the maximum allowable occupancy level of a given facility. In some states within the US, up to 50 people were allowed to participate in a program with proper physical distancing measures. For educational institutions, flexibility to allow 50% of the total capacity by following mandatory physical distancing and face mask measures has been considered in some states. Therefore, in the analysis, we considered two different occupancy levels (maximum 50 attendance, maximum 50% capacity) for different simulation configurations. The proposed seating also provides an additional feature of evaluating the maximum allowable occupancy by following mandatory physical distance and masking measures. The maximum allowable occupancy may vary for the same facility due to seating arrangements, the number of entrances and exits, and door locations.

The models were tested with different simulation configurations. However, it is more important to test different seating algorithms for each simulation configuration and obtain relevant statistics associated to the corresponding seating policy. The initial configuration of the simulation model is shown below:
i. Fixed Configurations:
   Facility type: Regular classroom
   Class occupancy: 264, Class duration: 50 min, Class time: 8:00 am.
ii. Experimented Configurations
   Layout: a. Collaborative, b. Traditional
   Exit rule: a. Zonal, b. Non zonal
   Maximum allowable attendance: a. 50% (132students) b. 19% (50 students)
   Seat choice: a. SD policy b. AS policy

Simulation Results

The simulation model was set up and data was analyzed for different configurations of classroom type, allowable occupancy, exit policy, and seating policies. In addition to the various exit and seat selection configurations, we have considered some fixed configurations for testing the policies and analyzing their impacts. Fixed configurations of the classroom include layout factors that could be used to decide the appropriate placement of desks. The model layout presented in this analysis include collaborative, and traditional seating arrangement. Statistics discussed and analyzed in this section for collaborative and traditional classroom settings are specific for the layout used in the simulation. However, the model framework presented in this analysis can be easily modified for different layout and thus can be utilized to study different indoor configurations.

Exit Time

Exit time is one of the most important factors in order to decide the appropriate break time to allow students of the previous class to safely exit and students of the next class to enter the classroom. Hence, the analysis of exit time for a different type of simulation configuration plays a vital role in the identification of the best policies. The boxplot in FIG. 28 conforms to the intuition of a lower exit time for 19% occupancy compared to the case of 50%. Collaborative classrooms work uniformly better than traditional settings except for the zonal exit with 19% occupancy. Interestingly, the exit time with traditional settings and 19% occupancy is not affected much by the exit policy compared to collaborative settings. That occurs due to the more streamlined movement of students in the traditional settings due to long desks, which ensures a natural queue during exit operation.

Average Exposure Duration

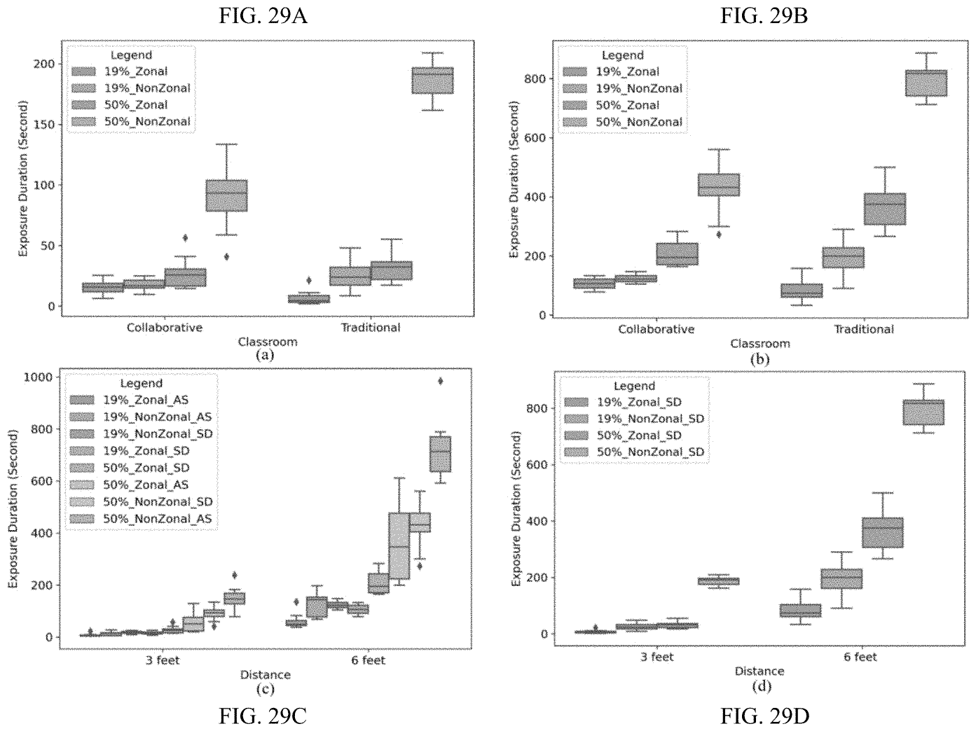

Safer operations of the indoor activities cannot be achieved without considering the exposure-related metrics. Hence, this analysis utilizes and evaluates the average exposure duration of each student within two distance ranges: 0-3, and 3-6 feet. The exposure durations have been calculated for different classroom settings, exit policies, and seat selection policies. As shown in FIG. 29A exposure duration at 0-3 feet and FIG. 29B shows exposure duration at 3-6 feet respectively, collaborative classrooms perform uniformly better than traditional settings at both distance ranges except for one combination (19% occupancy with zonal exit). Considering that combination, traditional classroom settings perform better due to the natural queue formed (guided by long desks) within the seating area. However, that benefit is less pronounced during higher occupancy (50%) and non-zonal exit policy due to more restricted pathways. With higher occupancy, wide and open pathways become more important to avoid congestion during non-zonal exit policy. Correspondingly, collaborative settings facilitate higher flexibility with more row opening towards the pathways, which helps the crowd to retract to their seats more easily compared to traditional settings during high congestion at the door/pathway areas. That eventually helps in reducing the exposure time in collaborative settings during high occupancy.

Exposure duration at different distance buckets (0-3 and 3-6 feet) acknowledge higher risk associated with a non-zonal exit policy. From FIG. 29C for collaborating settings and FIG. 29D for traditional settings, clearly at a lower occupancy level (19%), exposure duration is not much affected by exit and seating policies in 0-3 feet range. However, at the higher occupancy (50%) level, the zonal exit policy results in a considerably lower exposure duration compared to the non-zonal exit policy. That result validates pedestrian behavior where unregulated movement towards the exit door intuitively causes high exposure risk. Additionally, from FIGS. 29C and 29D, distinctively, the proposed SD seating policy causes a lower exposure duration at higher occupancy (50%) level. However, both SD and AS seating policies perform identically for lower occupancy (19%) level.

Average Number of Contacts

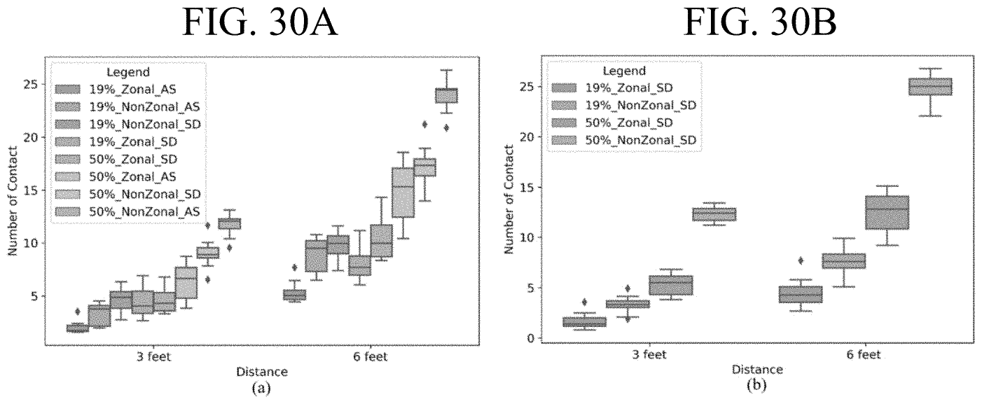

In addition to the average exposure duration, the average contact number provides insights pertaining to the number of other students within the range. It is important to parallelly analyze the average contact number because the exposure duration for each student shows the cumulative exposure time. Hence average contact number provides the distribution of exposure time within the proximity of each agent. As shown in FIG. 30A for collaborative settings and FIG. 30B for traditional settings, the zonal policy facilitates the significant reduction of the average contact number. Intuitively, a non-zonal exit policy causes unregulated outward flow and thus leads to higher physical distancing violation, which in turn would lead to a higher contact number. Hence, in order to ensure the safer operations of indoor activities, it is important to ensure the regulated flow of students using zonal exit policies. Additionally, at 50% occupancy, the proposed SD seating policy ensures a smaller number of contacts at all the distance range when the zonal exit policy is implemented. That occurs due to higher penalty at the pathway and door side seats which ensures minimum number of contacts between agents. However, at lower occupancy (19%), the AS seating policy performs better which goes against the intuition.

Implementation of zonal exit policy would lead to higher exit times as it increases the commute distance and time within the classroom. Hence, in terms of policy making the appropriate trade off needs to be established between the safety and commutation to ensure optimal operations of the indoor spaces.

The model was tested by changing the percentage of people who follow the 6 feet physical distancing guideline to mimic the real-world scenario where some people may break the regulations because of ignorance or unwillingness. Users can set the percentage of agents who will follow the physical distancing rule from the simulation dashboard startup screen. For physical distancing follower percentage, we tested four different combinations (i.e., 100 percent, 80 percent, 60 percent, and 40 percent). The value 80 percent indicates that 80 percent of the classroom participants will adhere to the strict physical distancing requirement, while the remaining 20 percent are not restricted by the rule. We considered a higher occupancy level (50%), as well as a collaborative classroom layout as a fixed simulation setting.

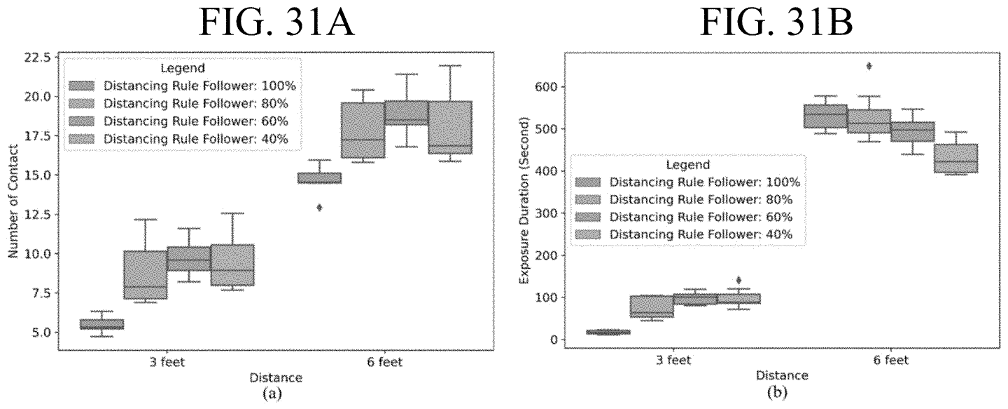

As demonstrated in FIG. 31A for a number of contacts and FIG. 31B for exposure duration in boxplots, we were able to make some interesting observations by varying the physical distancing follower percentage. When 100% of the students obey the rule, the value for both the number of contacts and the exposure duration is fairly low. If we lower the proportion to 80%, both the exposure duration and the number of contacts increased which is aligned with the intuition. However, if we decrease the percentage further (e.g., 60%, 40%), interestingly at 0-3 feet range number of contact and exposure duration does not change significantly. That behavior can be described from the point of view of a student's physical distancing circle who is supposed to be a rule follower. When everyone maintains the distance rule, no one, without exception, crosses the physical distancing circle (0-3 feet) of others (e.g., deadlock). However, if 20% of the students do not obey the rule, they may trespass into the distancing circle of another 20% of students (or more if the student interacts with multiple students at the very same time) who are willing to respect the rules. That may result in only 60% or less effective rule followers, despite the fact that the rule was modeled to be followed by 80% of the students. Similarly, if 60% of students are supposed to obey the rules, the number of effective rule followers will be less than planned. However, since 40% of students are not restricted by the rule, there will be a larger likelihood of contact amongst willing rule breakers. That could explain the possible equilibrium point in average contact number and contact time at the 60%, 40% or lower rule follower levels.

In contrast, there is a declining trend in exposure duration at 3-6 feet range as the rule breaker percentage rises. Some of the students who were previously at 3-6 feet range will get closer to each other and will be within 0-3 feet range as the percentage of rule followers decreases (e.g., from 100% to 80%). Because those contacts are now in the 0-3 feet time bucket, they will be removed from the 3-6 feet time bucket and the exposure duration for that distance will be reduced. Exit time for the students can also be used to explain this behavior. When the number of students who follow the rules is reduced from 100% to 80%, 60%, and 40%, students leave the classroom faster, resulting in less time spent in exit operation. Because of the less exit time, eventually students spend less time within 3-6-foot zone, and so exposure duration for that time bucket decreases.

Factor Effect Analysis

In this analysis, two independent variables namely, classroom occupancy and exit policy have been considered under four different scenarios to evaluate the traditional layout of the classroom. A significant positive correlation was observed based on the correlation analysis between the exposure duration and the number of contacts (r=0.97, p<0.01). Furthermore, multivariate analysis of variance (MANOVA) was studied in order to test the main and interaction effects with the exposure duration and contact distance under the traditional classroom layout. We observed that, the classroom occupancy has a significant main effect (F (1, 79)=124.54, p<0.01). Exit policies also demonstrated significant main effect (F (1, 79)=51.78, p<0.01). It is also evident that there is a significant interaction effect between the allowable occupancy and exit policy (F (1, 79)=21.49, p<0.01), which indicates the importance of exit policies given a higher occupancy percentage of the classroom.

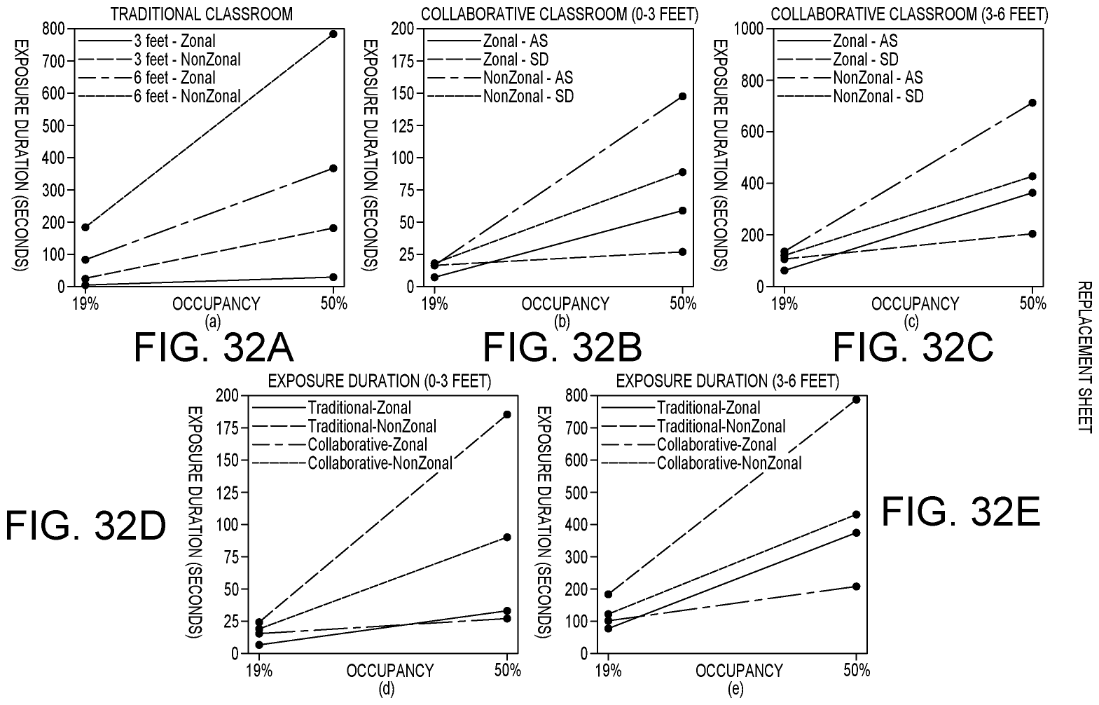

FIG. 32A shows the average exposure duration for traditional classroom layout within two distance ranges (0-3, and 3-6 feet) under different occupancy level. Hence, it can be inferred that exit policies shows significant differences when the maximum allowable occupancy of the classroom is higher. For collaborative classrooms, experiments were conducted to analyze the main effects and interaction effects using three different independent variables: classroom occupancy, exit policy, and seating policy. The response variable under consideration includes exposure duration, whereas contact distance was a covariate. Based on the correlation analysis, a significant positive correlation was observed between the number of contact and exposure duration (r=0.95, p<0.01). The multi-analysis of variance was conducted to test the main effects and interaction effects with the exposure duration. Significant main effects have been observed for classroom occupancy (F (1, 159)=138.20, p<0.01) and exit policy (F (1, 159)=40.17, p<0.01). Similarly, significant interaction effect was observed between the classroom occupancy and exit policy (F (1, 159)=21.99, p<0.01). Interestingly, a significant interaction effect between the occupancy and seating selection policy was also observed (F (1, 159)=19.54, p<0.01).

Based on the analysis for the collaborative classroom layout, it can be inferred that the exit policy and seat selection policy play a crucial role in ensuring the safer operation of classroom activities. As shown in FIG. 32B and FIG. 32C, significant reductions in the average exposure duration show that the zonal exit and the proposed SD seat selection policy makes a difference in the exposure, thus helpful in controlling the classroom infectious risk level under the collaborative classroom setting.

Another important aspect that needs to be investigated includes the type of classroom layout that a university should adopt given safety and logistics. Hence, the main effects and interaction effects for three factors namely, occupancy %, exit policy, and classroom type were tested to draw conclusions pertaining to the layout and policy requirements for the classroom operations. The three-way interaction effect was non-significant (F (1,159)=2.49, p>0.05). However, a significant interaction effect between classroom type and occupancy was observed (F (1, 159)=25.80, p<0.01), as shown in FIGS. 32D and 32E.

Verification and Validation

The verification and validation is an iterative process that takes place throughout the development phase of any simulation analysis. Because the simulation models developed during this analysis represent actual university facilities (e.g., classroom, meeting room, auditorium, office room, dance class), significant time was spent visiting all of space, and documenting functional specifications (e.g., type of indoor space, number of doors, total capacity, room layout, class time, break time) and spatial information (e.g., facility size, number of desks and chairs, the gap between rows and columns, door position, teacher's corner position). Furthermore, the analysis used the University of Arizona's “Interactive Floorplans” platform to assess the spatial position of a facility, the number of connecting hallways and hallway dimensions, the number of floors, and the location of the staircase to accurately model incoming and outgoing pedestrians. Throughout the model development process, the models were constantly presented to the university facility management and the campus reopening authority to verify the collected data and solicit suggestions for model parameters and policy evaluation (e.g., entrance and exit policy, seating policy, classroom occupancy policy).

As decision-makers and individuals intend to use the developed models to evaluate their decisions, they are highly concerned about the accuracy of the models. In this analysis, there were two types of results: a. mimicking real-world pedestrian dynamics and b. statistics (i.e., exit time, number of contacts, exposure duration). As previously stated, the developed models were constantly presented to the university facility management personnel, the campus reopening authority, a group of students and research experts to verify and validate them and replicate realistic pedestrian movement within the indoor space. During the verification and validation process, valuable feedbacks were provided regarding certain irregular crowd motions, which we later identified as deadlock situations and resolved by implementing the deadlock detection and resolution algorithm. After several iterations, the model development was completed, which was then used by stakeholders to evaluate different configurations of the indoor facility.

Due to university restrictions on in-person class attendance, testing the simulation statistics against any real data or recorded video during the COVID-19 pandemic was not possible. However, due to extensive verification and validation efforts with the campus experts as well as availability of highly detailed data of the indoor facilities, the developed models are believed to be sufficiently valid and accurate to provide meaningful managerial insights in evaluating alternative policies and scenarios.

This analysis can serve as a foundation to incorporate disease propagation based on contact-caused risk within an indoor facility. That will facilitate in getting deeper insights on hotspots throughout the facility and identify the high-risk areas. The physical distancing and deadlock resolution mechanisms have been considered in this analysis to incorporate a high degree of realism in pedestrian behaviors. Furthermore, we have conducted model testing under different entrance and exit policies, seating policies to reduce the contacts and exposure due to physical distancing violations. Different policies were assessed based on outputs depicting the logistics (e.g., exit time) and risk matrices (e.g., number of contact and exposure duration). Based on the simulation results, it was suggested that utilizing a collaborative classroom with zonal exit policies leads to the significant reduction of exposure risk in a higher occupancy level. Moreover, implementing the proposed social distancing seat assignment approach played a crucial role in reducing the exposure duration. However, with a significantly lower occupancy level, the classroom layout did not play a significant role in reducing exposure risk levels.

This application can be used in K-12 schools across the country to ensure minimal student contact and a safer classroom environment. Furthermore, institutions (e.g., schools, colleges, universities, offices), business owners (e.g., restaurants, groceries), and prayer hall authorities (e.g., mosque, church) can conduct different what-if analyses by rearranging the chairs, desks, and walkways to determine the best seating arrangement in terms of minimum contacts within the indoor space. Because of the high-fidelity simulation videos, this application may also be used to teach people how to properly enter, take a seat, and exit an indoor facility while adhering to policy (e.g., zonal vs. non-zonal, physical distancing). That analysis can provide some basic takeaways for organizations that do not have the expertise to use this modeling technology. One is to implement SD seating policy, which can be readily accomplished by ensuring that the seats with the greatest distance from the doors are the first to be occupied, reducing recurrent contact between persons sitting near the doors. By designing zones in a large interior space, organizations may ensure a zonal exit strategy. Furthermore, everyone should conform to the physical distancing rules because if some students do not, they may come into contact with others who wish to follow the rules, lowering the effective rule follower percentage significantly.

Detailed analysis of disease propagation within indoor spaces can be performed by incorporating the droplet and airborne transmission models. Key factors affecting droplet and airborne transmission, including breathing rate, agent's height and weight, classroom volume, class length, HVAC parameters, and mask-wearing percentages, would undoubtedly enhance the modeling and analysis capabilities within indoor spaces during a pandemic situation. Furthermore, while this analysis only considered a 3-feet physical distancing policy, this framework can be modified to incorporate different physical distancing recommendations based on the requirements. Although the policy choice depends on different factors, it is worth investigating to find the best combination of policies to make indoor safer in this pandemic situation. The outputs from the analysis can be used to fit an appropriate meta-model, which can provide relevant input parameters for an organization-wide disease propagation model. Moreover, the framework can be deployed to assess operations in other organizations such as manufacturing facilities, hospitals, and military bases. Additional features into the system can be incorporated to provide an online decision support system for different stakeholders to provide the real-time assessment of the situation in particular indoor spaces.

### REFERENCES

Anylogic, (retrieved from 27/09/2020), GIS Models. Anylogic Help. https://help.anylogic.com/index.jsp?topic=%2Fcom.anylogic.help%2Fhtml%2Fgis%2Fgis-architecture.html
Centers for Disease Control and Prevention, (retrieved from 10/09/2020), COVID-19 Pandemic Planning Scenarios. https://www.cdc.gov/coronavirus/2019-ncov/hcp/planning-scenarios.html
Oran, D. P., & Topol, E. J. (2020). Prevalence of Asymptomatic SARS-CoV-2 Infection: A Narrative Review. Annals of Internal Medicine.
Lauer, S. A., Grantz, K. H., Bi, Q., Jones, F. K., Zheng, Q., Meredith, H. R., . . . & Lessler, J. (2020). The incubation period of coronavirus disease 2019 (COVID-19) from publicly reported confirmed cases: estimation and application. Annals of internal medicine, 172(9), 577-582.
Tindale, L., Coombe, M., Stockdale, J. E., Garlock, E., Lau, W. Y. V., Saraswat, M., . . . & Colijn, C. (2020). Transmission interval estimates suggest pre-symptomatic spread of COVID-19. MedRxiv.
Zhu, C. Q., Gao, S. D., Xu, Y., Yang, X. H., Ye, F. Q., Ai, L. L., . . . & Liu, Y. S. (2020). A COVID-19 case report from asymptomatic contact: implication for contact isolation and incubation management. Infectious diseases of poverty, 9(1), 1-7.
Seow, J., Graham, C., Merrick, B., Acors, S., Steel, K. J., Hemmings, O., & Betancor, G. (2020). Longitudinal evaluation and decline of antibody responses in SARS-CoV-2 infection. MedRxiv.
Centers for Disease Control and Prevention, (retrieved from 10/09/2020), Interim Clinical Guidance for Management of Patients with Confirmed Coronavirus Disease (COVID-19). https://www.cdc.gov/coronavirus/2019-ncov/hcp/clinical-guidance-management-patients.html
Centers for Disease Control and Prevention, (retrieved from 04/09/2020), Interim Guidance for Rapid Antigen Testing for SARS-CoV-2. https://www.cdc.gov/coronavirus/2019-ncov/lab/resources/antigen-tests-guidelines.html
Woloshin, S., Patel, N., & Kesselheim, A. S. (2020). False Negative Tests for SARS-CoV-2 Infection—Challenges and Implications. New England Journal of Medicine.
The University of Arizona, (retrieved from 27/09/2020). Events & Gathering. https://covid19.arizona.edu/reentry-plan/events
The University of Arizona, (retrieved from 15/09/2020). Shelter-in-place self-quarantine. https://uapd.arizona.edu/news/2020/09/shelter-place-self-quarantine
The University of Arizona, (retrieved from 27/09/2020). Face Coverings. https://covid19.arizona.edu/face-coverings
The University of Arizona, (retrieved from 27/09/2020). Test, Trace, Treat. https://covid19.arizona.edu/test-trace-treat
The University of Arizona, (retrieved from 27/09/2020). Self-Quarantine Guidelines. https://covid19.arizona.edu/self-quarantine
Watanabe, T., Bartrand, T. A., Weir, M. H., Omura, T., & Haas, C. N. (2010). Development of a dose-response model for SARS coronavirus. Risk Analysis: An International Journal, 30(7), 1129-1138.
Wilson, A. M., Aviles, N., Beamer, P. I., Szabo, Z., Ernst, K. C., & Masel, J. (2020). Quantifying SARS-CoV-2 infection risk within the Apple/Google exposure notification framework to inform quarantine recommendations. medRxiv.
Eikenberry, S. E., Mancuso, M., Iboi, E., Phan, T., Eikenberry, K., Kuang, Y., . . . & Gumel, A. B. (2020). To mask or not to mask: Modeling the potential for face mask use by the general public to curtail the COVID-19 pandemic. Infectious Disease Modelling.
Jimenez (2020), https://cires.colorado.edu/news/covid-19-airborne-transmission-tool-available
Morawska, L., Tang, J. W., Bahnfleth, W., Bluyssen, P. M., Boerstra, A., Buonanno, G., . . . & Haworth, C. (2020). How can airborne transmission of COVID-19 indoors be minimised?. Environment international, 142, 105832.
Wang, Y., Tian, H., Zhang, L., Zhang, M., Guo, D., Wu, W., . . . & Liu, B. (2020). Reduction of secondary transmission of SARS-CoV-2 in households by face mask use, disinfection and social distancing: a cohort study in Beijing, China. BMJ Global Health, 5(5), e002794.
COVID-19 Projections Using Machine Learning, (retrieved from 27/09/2020). Covid-19 projection in Arizona. https://covid19-projections.com/us-az
Centers for Disease Control and Prevention, (retrieved from 07/07/2020), Considerations for Events and Gatherings. https://www.cdc.gov/coronavirus/2019-ncov/community/large-events/considerations-for-events-gatherings.html
https://www.who.int/dg/speeches/detail/who-director-general-s-opening-remarks-at-the-media-briefing-on-covid-19---3-august-2020
Helbing, D., Farkas, I., & Vicsek, T. (2000). Simulating dynamical features of escape panic. Nature, 407(6803), 487-490.
Salari, M., Milne, R. J., Delcea, C., Kattan, L., & Cotfas, L. A. (2020). Social distancing in airplane seat assignments. Journal of Air Transport Management, 89, 101915.
https://www.aappublications.org/news/2020/06/26/schoolreopening062620#:˜:text=Social%20distancing%2C%20mask%20use&text=%E2%80%9CEvidence%20suggests%20that%20spacing%20as,%2C%E2%80%9D%20according%20to%20the%20guidance.
“COVID-19 Map—Johns Hopkins Coronavirus Resource Center.” https://coronavirus.jhu.edu/map.html (accessed Feb. 2, 2021).
The New York Times, “Tracking the Coronavirus at U.S. Colleges and Universities—The New York Times,” Dec. 11, 2020. https://www.nytimes.com/interactive/2020/us/covid-college-cases-tracker.html (accessed Feb. 2, 2021).
A. A. Weil et al., “SARS-CoV-2 Epidemiology on a Public University Campus in Washington State,” medRxiv, p. 2021.03.15.21253227, January 2021, doi: 10.1101/2021.03.15.21253227.
K. E. Vang et al., “Participation in Fraternity and Sorority Activities and the Spread of COVID-19 Among Residential University Communities—Arkansas, Aug. 21-Sep. 5, 2020,” MMWR. Morb. Mortal. Wkly. Rep., vol. 70, no. 1, pp. 20-23, January 2021, doi: 10.15585/mmwr.mm7001a5.
E. Wilson et al., “Multiple COVID-19 Clusters on a University Campus—North Carolina, August 2020,” MMWR. Morb. Mortal. Wkly. Rep., vol. 69, no. 39, pp. 1416-1418, October 2020, doi: 10.15585/mmwr.mm6939e3.
Centers for Disease Control and Prevention, “Operational Considerations for Schools,” US Departement of Health and Human Service, 2020. https://www.cdc.gov/coronavirus/2019-ncov/global-covid-19/schools.html#:˜:text=Instruct students to maintain at, together and during class breaks.
H. Melnick et al., “Reopening schools in the context of COVID-19: Health and safety guidelines from other countries,” Learn. Policy Inst., no. May, pp. 1-13, 2020, [Online]. Available: https://learningpolicyinstitute.org/sites/default/files/product-files/Reopening_Schools_COVID-19_BRIEF.pdf.
N. G. Davies et al., “Age-dependent effects in the transmission and control of COVID-19 epidemics,” Nat. Med., vol. 26, no. 8, pp. 1205-1211, August 2020, doi: 10.1038/s41591-020-0962-9.
P. N. Zivich et al., “Transmission of viral pathogens in a social network of university students: the eX-FLU study,” Epidemiol. Infect., vol. 148, p. e267, August 2020, doi: 10.1017/S0950268820001806.
N. Hoertel et al., “Facing the COVID-19 epidemic in NYC: A stochastic agent-based model of various intervention strategies,” medRxiv. medRxiv, Apr. 28, 2020, doi: 10.1101/2020.04.23.20076885.
B. Lopman et al., “A model of COVID-19 transmission and control on university campuses,” medRxiv, p. 2020.06.23.20138677, January 2020, doi: 10.1101/2020.06.23.20138677.
A. Elbanna et al., “Entry screening and multi-layer mitigation of COVID-19 cases for a safe university reopening,” medRxiv, p. 2020.08.29.20184473, January 2020, doi: 10.1101/2020.08.29.20184473.
D. R. E. Ranoa et al., “Saliva-Based Molecular Testing for SARS-CoV-2 that Bypasses RNA Extraction,” bioRxiv, p. 2020.06.18.159434, January 2020, doi: 10.1101/2020.06.18.159434.
D. M. Goldfarb et al., “Self-collected Saline Gargle Samples as an Alternative to Healthcare Worker Collected Nasopharyngeal Swabs for COVID-19 Diagnosis in Outpatients,” medRxiv, p. 2020.09.13.20188334, January 2020, doi: 10.1101/2020.09.13.20188334.
Centers for Disease Control and Prevention, “Interim Clinical Considerations for Use of COVID-19 Vaccines CDC.” https://www.cdc.gov/vaccines/covid-19/info-by-product/clinical-considerations.html (accessed Apr. 9, 2021).
G. Pandey, P. Chaudhary, R. Gupta, and S. Pal, “SEIR and Regression Model based COVID-19 outbreak predictions in India,” April 2020, Accessed: Apr. 10, 2021. [Online]. Available: https://arxiv.org/abs/2004.00958.
S. He, Y. Peng, and K. Sun, “SEIR modeling of the COVID-19 and its dynamics,” Nonlinear Dyn., vol. 101, no. 3, pp. 1667-1680, 2020, doi: 10.1007/s11071-020-05743-y.
S. Mwalili, M. Kimathi, V. Ojiambo, D. Gathungu, and R. Mbogo, “SEIR model for COVID-19 dynamics incorporating the environment and social distancing,” BMC Res. Notes, vol. 13, no. 1, p. 352, 2020, doi: 10.1186/s13104-020-05192-1.
P. van den Driessche, “Reproduction numbers of infectious disease models,” Infect. Dis. Model., vol. 2, no. 3, pp. 288-303, June 2017, doi: 10.1016/j.idm.2017.06.002.
C. C. John, V. Ponnusamy, S. K. Chandrasekaran, and R. N., “A Survey on Mathematical, Machine Learning and Deep Learning Models for COVID-19 Transmission and Diagnosis,” IEEE Rev. Biomed. Eng., p. 1, 2021, doi: 10.1109/RBME.2021.3069213.
N. S. Punn, S. K. Sonbhadra, and S. Agarwal, “COVID-19 Epidemic Analysis using Machine Learning and Deep Learning Algorithms,” medRxiv, p. 2020.04.08.20057679, January 2020, doi: 10.1101/2020.04.08.20057679.
M. Li et al., “Identifying novel factors associated with COVID-19 transmission and fatality using the machine learning approach,” Sci. Total Environ., vol. 764, p. 142810, 2021, doi: https://doi.org/10.1016/j.scitotenv.2020.142810.
D. Efimov and R. Ushirobira, “On an interval prediction of COVID-19 development based on a SEIR epidemic model,” Annu. Rev. Control, 2021, doi: https://doi.org.10.1016/j.arcontrol.2021.01.006.
R. Dandekar and G. Barbastathis, “Neural Network aided quarantine control model estimation of global Covid-19 spread,” April 2020, Accessed: Apr. 10, 2021. [Online]. Available: http://arxiv.org/abs/2004.02752.
E. Cuevas, “An agent-based model to evaluate the COVID-19 transmission risks in facilities,” Comput. Biol. Med., vol. 121, p. 103827, 2020, doi: https://doi.org.10.1016/j.arcontrol.2020.103827.
N. Hoertel et al., “A stochastic agent-based model of the SARS-CoV-2 epidemic in France,” Nat. Med., vol. 26, no. 9, pp. 1417-1421, 2020, doi: 10.1038/s41591-020-1001-6.
N. Mandizadeh Gharakhanlou and N. Hooshangi, “Spatio-temporal simulation of the novel coronavirus (COVID-19) outbreak using the agent-based modeling approach (case study: Urmia, Iran),” Informatics Med. Unlocked, vol. 20, p. 100403, January 2020, doi: 10.1016/j.imu.2020.100403.
I. Mahmood et al., “FACS: a geospatial agent-based simulator for analysing COVID-19 spread and public health measures on local regions,” J. Simul., pp. 1-19, August 2020, doi: 10.1080/17477778.2020.1800422.
Magzhan, K., Jani, H. M. (2013) “A Review and Evaluations of Shortest Path Algorithm”, International Journal of Scientific & Technology Research Volume 2, Issue 6.
M. T. Islam, S. Jain, Y. Chen, B. Barua Dripta, and Y.-J. Son, “An Agent Based Simulation Model to Evaluate Contacts, Layout, and Policies in Entrance, Exit, and Seating in Indoor Activities Under a Pandemic Situation,” 2021. Manuscript submitted for publication.
N. I. Stilianakis and Y. Drossinos, “Dynamics of infectious disease transmission by inhalable respiratory droplets,” J. R. Soc. Interface, vol. 7, no. 50, pp. 1355-1366, September 2010, doi: 10.1098/rsif.2010.0026.
T. Watanabe, T. A. Bartrand, M. H. Weir, T. Omura, and C. N. Haas, “Development of a Dose-Response Model for SARS Coronavirus,” Risk Anal., vol. 30, no. 7, pp. 1129-1138, July 2010, doi: https://doi.org/10.1111/j.1539-6924.2010.01427.x.
S. K. Halloran, A. S. Wexler, and W. D. Ristenpart, “A Comprehensive Breath Plume Model for Disease Transmission via Expiratory Aerosols,” PLoS One, vol. 7, no. 5, p. e37088, May 2012, [Online]. Available: https://doi.org/10.1371/journal.pone.0037088.
X. Xie, Y. Li, A. T. Y. Chwang, P. L. Ho, and W. H. Seto, “How far droplets can move in indoor environments—revisiting the Wells evaporation—falling curve.,” Indoor Air, vol. 17, no. 3, pp. 211-225, June 2007, [Online]. Available: http://10.0.4.87/j.1600-0668.2007.00469.x.
“COVID-19 Airborne Transmission Tool Available|CIRES.” https://cires.colorado.edu/news/covid-19-airborne-transmission-tool-available (accessed Apr. 11, 2021).
S. L. Miller et al., “Transmission of SARS-CoV-2 by inhalation of respiratory aerosol in the Skagit Valley Chorale superspreading event,” Indoor Air, vol. 31, no. 2, pp. 314-323, March 2021, doi: https://doi.org/10.111/ina.12751.
Y. Wang et al., “Reduction of secondary transmission of SARS-CoV-2 in households by face mask use, disinfection and social distancing: a cohort study in Beijing, China,” BMJ Glob. Heal., vol. 5, no. 5, p. e002794, May 2020, doi: 10.1136/bmjgh-2020-002794.
A. Scohy, A. Anantharajah, M. Bodéus, B. Kabamba-Mukadi, A. Verroken, and H. Rodriguez-Villalobos, “Low performance of rapid antigen detection test as frontline testing for COVID-19 diagnosis,” J. Clin. Virol., vol. 129, p. 104455, 2020, doi: https://doi.org/10.1016/j.jcv.2020.104455.
J. Dinnes et al., “Rapid, point-of-care antigen and molecular-based tests for diagnosis of SARS-CoV-2 infection,” Cochrane Database Syst. Rev., no. 3, 2021, doi: 10.1002/14651858.CD013705.pub2.
“How the SARS-CoV-2 EUA Antigen Tests Work.” https://asm.org/Articles/2020/August/How-the-SARS-CoV-2-EUA-Antigen-Tests-Work (accessed Apr. 11, 2021).
J. Hellewell et al., “Estimating the effectiveness of routine asymptomatic PCR testing at different frequencies for the detection of SARS-CoV-2 infections,” medRxiv, p. 2020.11.24.20229948, January 2020, doi: 10.1101/2020.11.24.20229948.
T. Pilishvili, “Interim Estimates of Vaccine Effectiveness of Pfizer-BioNTech and Moderna COVID-19 Vaccines Among Health Care Personnel—33 U.S. Sites, January—March 2021,” MMWR. Morb. Mortal. Wkly. Rep., vol. 70, no. 20, pp. 753-758, 2021, doi: 10.15585/MMWR.MM7020E2.

The foregoing description and drawings should be considered as illustrative only of the principles of the invention. All references cited herein are incorporated in their entireties. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A method of predictive modeling, comprising the steps of:
receiving campus data, mobility data, disease propagation data, testing data, and policy data for a plurality of agents;
assigning a parameter setting and a profile to each of the plurality of agents;
executing movement of the plurality of agents in a map;
determining infectious risk and testing results for the plurality of agents;
updating the agent status for the plurality of agents; and
outputting the simulation results to a graphic user interface.

2. The method of claim 1, wherein movement of the plurality of agents is executed on a geographic information service (GIS) map.

3. The method of claim 1, wherein an event is comprised of a party on the weekend, manual contact tracing, a shelter-at-home policy, a social distancing policy, an indoor mask requirement policy, and a regular test policy.

4. The method of claim 1, wherein each agent's profile is comprised of the agent's daily schedule, location, periodic test information, and initial disease state.

5. The method of claim 1, further comprising predicting the positive rate and the positive test result rate for a disease among the plurality of agents.

6. The method of claim 1, wherein infectious risk is determined using a droplet transmission model that incorporates respiratory droplet aerodynamics.

7. The method of claim 1, wherein the profile for each of the plurality of agents incorporates an indoor movement model comprised of pedestrian dynamics with embedded social force.

8. A system for predictive modeling, wherein a server:
receives campus data, mobility data, disease propagation data, testing data, and policy data for a plurality of agents;
assigns a parameter setting and a profile to each of the plurality of agents;
executes movement of the plurality of agents in a map;
determines infectious risk and testing results for the plurality of agents;
updates the agent status for the plurality of agents; and
outputs the simulation results to a graphic user interface.

9. The system of claim 8, wherein movement of the plurality of agents is executed on a geographic information service (GIS) map.

10. The system of claim 8, wherein an event is comprised of a party on the weekend, manual contact tracing, a shelter-at-home policy, a social distancing policy, an indoor mask requirement policy, and a regular test policy.

11. The system of claim 8, wherein each agent's profile is comprised of the agent's daily schedule, location, periodic test information, and initial disease state.

12. The system of claim 8, wherein the server further predicts the positive rate and the positive test result rate for a disease among the plurality of agents.

13. The system of claim 8, wherein infectious risk is determined using a droplet transmission model that incorporates respiratory droplet aerodynamics.

14. The system of claim 8, wherein the profile for each of the plurality of agents incorporates an indoor movement model comprised of pedestrian dynamics with embedded social force.

15. A method of predictive modeling, comprising the steps of:
receiving facility parameters, agent parameter settings, and agent generation data for a plurality of agents;
calculating routing and seating policies for the plurality of agents;
calculating movement based on the self-consciousness of the agents, the force of other agents, and the force from the environment on the plurality of agents; and
determining an exit path restriction policy or a zonal policy for an enclosed area that minimizes the risk of disease propagation for the plurality of agents.

16. The system of claim 15, wherein the facility parameters are comprised of: capacity, policy, number of entry and exits, area of the location, and dimensions of the location.

17. The method of claim 15, wherein the agent parameter settings are comprised of velocity and diameter.

18. The method of claim 15, wherein the agent generation data is comprised of an arrival schedule and an arrival rate.

19. The method of claim 15, wherein the routing and seating policies are comprised of a shortest path analysis or a least cost analysis.

20. The method of claim 15, wherein movement is calculated using a deadlock detection and resolution process.
